(19) United States
(12) Patent Application Publication
(10) Pub. No.: US 2003/0170659 A1
(43) Pub. Date: Sep. 11, 2003

(54) Electrical treatment of binding media to encourage, discourage and/or study biopolymer binding

(75) Inventors: Glen H. Erikson, Providenciales (TC); Jasmine I. Daksis, Richmond Hill (CA)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(73) Assignee: Ingeneus Corporation, Bridgetown (BB)

(21) Appl. No.: 10/189,211

(22) Filed: Jul. 3, 2002

Related U.S. Application Data
(63) Continuation-in-part of application No. 10/120,092, filed on Apr. 10, 2002.
Application No. 10/120,092 is a continuation-in-part of application No. 09/911,047, filed on Jul. 23, 2001.
Application No. 09/911,047 is a continuation-in-part of application No. 09/490,273, filed on Jan. 24, 2000, now Pat. No. 6,265,170.
(63) Continuation-in-part of application No. 09/998,155, filed on Nov. 29, 2001.
Application No. 09/998,155 is a continuation-in-part of application No. 09/911,047, filed on Jul. 23, 2001.
Application No. 09/911,047 is a continuation-in-part of application No. 09/490,273, filed on Jan. 24, 2000, now Pat. No. 6,265,170.

(51) Int. Cl. C12Q 1/68

(52) U.S. Cl. 435/6; 205/777.5

(57) ABSTRACT

A method for influencing binding of a first biopolymer to a second biopolymer includes applying an electric charge to a binding medium in which the first and second biopolymers are to be bonded together, wherein the electric charge is applied sufficiently to enhance or diminish a binding characteristic of the binding to thereby enhance the binding desired, provided that the binding characteristic is not denaturation of the first and second biopolymers from each other or from another biopolymer.

### BACKGROUND OF THE INVENTION

1. Field of the Invention

The invention relates to methods and apparatus for encouraging, discouraging and/or studying biopolymer binding, and more particularly to such methods and apparatus based on the electrical treatment of the medium containing biopolymers.

2. Description of Related Art

It has been understood for a number of years that biological molecules can be isolated and characterized through the application of an electric field to a sample.

Electrophoresis is perhaps the most well known example of an isolation and characterization technique based on the influence of electric fields on biological molecules. In gel electrophoresis, a uniform matrix or gel is formed of, for example, polyacrylamide, to which an electric field is applied. Mixtures applied to one end of the gel will migrate through the gel according to their size and interaction with the electric field. Mobility is dependent upon the unique characteristics of the substance such as conformation, size and charge. Mobilities can be influenced by altering pore sizes of the gel, such as by altering the concentration of the acrylamide, bis-acrylamide, agarose or cross-linking agent, or by formation of a concentration or pH gradient, or by altering the composition of the buffer (pH, SDS, DOC, glycine, salt). One- and two-dimensional gel electrophoresis are fairly routine procedures in most research laboratories. Target substances can be purified by passage through and/or physical extraction from the gel.

A more recently developed process in which an electric field is applied to a biological sample is disclosed in U.S. Pat. No. 5,824,477 to Stanley. The Stanley patent discloses a process for detecting the presence or absence of a predetermined nucleic acid sequence in a sample. The process comprises: (a) denaturing a sample double-stranded nucleic acid by means of a voltage applied to the sample in a solution by means of an electrode; (b) hybridizing the denatured nucleic acid with an oligonucleotide probe for the sequence; and (c) determining whether the hybridization has occurred. The Stanley patent discloses the application of an electric field to the sample to be assayed for the limited purpose of denaturing the target sequence.

It must be understood that when “hybridization” is used herein it refers to all the possible varieties of specific nucleic acid binding, such as triplex and quadruplex binding, and is not reserved to describe duplex binding only.

A better-known type of hybridization assay is based on the use of fluorescent marking agents. In their most basic form, fluorescent intensity-based assays have typically comprised contacting a target with a fluorophore-containing probe, removing any unbound probe from bound probe, and detecting fluorescence in the washed sample. Homogeneous assays improve upon such basic assays, in that the former do not require a washing step or the provision of a non-liquid phase support.

In the ongoing search for more sensitive, accurate and rapid assay techniques, one research group developed an assay comprising analyzing the effects of an electric field on the fluorescent intensity of nucleic acid hybridization duplexes. See U.S. patent application Ser. No. 08/807,901, filed Feb. 27, 1997 and U.S. Pat. No. 6,060,242. The researchers indicated that the fluorescent intensity emitted from a one base-pair mismatched duplex differed from that emitted from a perfectly matched duplex. U.S. Pat. No. 6,265,170 discloses a method for detecting a nucleotide sequence, wherein an electric field is applied to a liquid medium prior to or concurrently with a detecting step, wherein the electric field causes a change in an intensity of a fluorescent emission which indicates whether the probe is hybridized to a completely complementary nucleotide sequence or an incompletely complementary nucleotide sequence.

Despite the foregoing developments, it remains desirable to provide new methods for influencing the binding properties of biopolymers such as nucleic acids. Such methods provide diagnostics that display improved characteristics as well as provide new tools for analysis and understanding of biopolymer activity. Improved therapeutics are also foreseen.

It is also desired to provide a simple, highly sensitive, effective and rapid method for analyzing interaction between nucleic acids and/or nucleic acid analogues.

It is further desired to provide a novel apparatus for encouraging, discouraging and/or assaying binding of probes and targets containing nucleobases and/or amino acids.

It is still further desired to provide such a novel apparatus, wherein binding is assayed by measuring optical and/or electrical properties of a sample.

All references cited herein are incorporated herein by reference in their entireties.

### BRIEF SUMMARY OF THE INVENTION

Accordingly, the invention provides a method for influencing binding of a first biopolymer to a second biopolymer, said method comprising applying an electric charge to a binding medium in which the first and second biopolymers are to be bonded together, wherein the electric charge is applied sufficiently to enhance or diminish a binding characteristic of the binding to thereby influence the binding, provided that the binding characteristic is not denaturation of the first and second biopolymers from each other or from another biopolymer.

Also provided is an apparatus for performing the method of the invention. The apparatus comprises a binding medium container, electrodes adapted to contact the binding medium in the binding medium container, a voltage source to apply the electric charge to the binding medium in the binding medium container and a detector.

### BRIEF DESCRIPTION OF SEVERAL VIEWS OF THE DRAWINGS

The invention will be described in conjunction with the following drawings in which like reference numerals designate like elements and wherein:

FIGS. 1A, 1B, 2A and 2B are graphs of fluorescent intensity as a function of time and applied voltage;

FIGS. 3A, 3B, 4A, 4B, 5A, 5B, 6A, 6B, 7, 8A, 8B, 8C and 8D are fluorescent intensity spectra;

FIGS. 9A and 9B are graphs of fluorescent intensity as a function of time and applied voltage.

FIG. 10 is an overhead schematic view of an embodiment of an apparatus of the invention;

FIG. 11 is a partial cross-sectional view through line 11-11 of FIG. 10;

FIG. 12 is a schematic view of another embodiment of the apparatus of the invention; and

FIG. 13 is a schematic view of another embodiment of the apparatus of the invention.

### DETAILED DESCRIPTION OF THE INVENTION

The invention is based on our discovery that electric treatment of a medium containing (or that will contain) biopolymers, such as nucleic acids and/or analogues thereof, can be used to influence the binding which occurs within the medium. A variety of binding characteristics can be adjusted through the application of electricity to the binding medium.

We have previously investigated the transitory effect of applying voltage to a test medium for purposes of evaluating the binding of reagents. We elucidated (e.g., in U.S. patent application Ser. No. 09/911,047, filed Jul. 23, 2001) that it was possible to select voltages that would cause a detectable change in a fluorescent signal emitted from an imperfectly bound nucleic acid complex, but not a perfectly matched nucleic acid complex.

We have previously discovered and investigated the binding motif preference of nucleic acid sequences and have elucidated facts that introduce a new level of complexity into the interpretation of signals emitted by reagent binding.

Binding Method

The present invention provides methods which will allow practitioners to enhance the speed and specificity of binding reactions and to identify signals arising from binding reactions otherwise permitted by the binding conditions which obscure the binding reactions which are to be evaluated.

Remarkably, we have discovered that there are methods of applying energy to a medium that will effect desirable gains in binding rate and specificity and even create conditions of binding motif selection, while leaving unchanged all other conditions of temperature, pH and the like of the medium and reagents.

In certain embodiments, the effect that we elucidate herein is progressive and proceeds to a maximum over a period of time at room temperature. Data disclosed in the Examples below show that the medium can achieve a maximum or saturation in about 20 minutes.

Our experimentation has shown that practice of the method of electrical treatment will have broadly varying effects based upon the voltage selected, the manner of its application, the presence of one or more reagents in the medium at the time of treatment, and the binding potential of the reagents, among other factors.

Remarkably, medium, once treated by the method, maintains its changed character even after transfer from a pretreatment container (e.g., a large vessel containing a stock solution) to another container (e.g., a small vessel) in which or on which the binding reaction is to be conducted.

The binding method of the invention is suitable for enhancing, hindering and/or studying the binding of biopolymers. The term “biopolymers” as used herein means a sequence containing at least two amino acids, amino acid analogues, nucleic acids, nucleic acid analogues and/or combinations thereof. Preferably, the biopolymers are nucleic acids or analogues thereof.

In certain embodiments in which first and second biopolymers are to be bonded together, the first biopolymer is a nucleic acid (preferably DNA or RNA) and the second biopolymer is a nucleic acid analogue (preferably PNA, LNA or another uncharged, partially charged or positively charged nucleic acid analogue).

As mentioned above, the invention can enhance binding of a first biopolymer to a second biopolymer by enhancing a binding characteristic. The binding characteristic can be, e.g., a specific binding affinity between the first and second biopolymers, which is increased by the electric charge as evidenced by an accelerated rate of binding, enhanced specificity, or both.

When the first and second biopolymers are nucleic acids or nucleic acid analogues capable of binding together in a Watson-Crick binding motif and in a homologous binding motif, the binding characteristic can be a binding motif preference, which is adjusted by the electric charge. For example, the electric charge can shift the binding motif preference towards the homologous binding motif sufficiently that the first and second biopolymers are solely or predominantly bound in the homologous binding motif, or shift the binding motif preference towards the Watson-Crick binding motif sufficiently that the first and second biopolymers are solely or predominately bound in the Watson-Crick binding motif.

The invention can also enhance binding of a first biopolymer to a second biopolymer by hindering a binding characteristic, such as a binding affinity between at least one of the first and second biopolymers and an object, wherein the binding affinity is reduced by the electric treatment of the binding medium.

There are a number of suitable ways by which the electric charge can be applied to the binding medium.

The timing of the electric charge application can vary. For example, the electric charge can be applied to the binding medium: (a) prior to introducing the first and second biopolymers therein; (b) when only one of the first and second biopolymers is present in the binding medium; and/or (c) when both the first and second biopolymers are present in the binding medium.

The electric charge can be applied to the binding medium in a first vessel, before the binding medium is transferred from the first vessel to a second vessel in which the first and second biopolymers are to be bonded together. Similarly, the electric charge can be applied to only a portion of the binding medium. In alternative embodiments, the electric charge can be applied to the entire binding medium.

The electric charge can be, e.g., AC or DC electrical current.

The electric charge can be applied in a single application or in multiple pulses. In pulsed embodiments, the pulses can be: (a) of varied currents; (b) applied for varied durations; and/or (c) applied at varied intervals. Preferably, the electric charge is applied as a plurality of DC pulses from 1 V to 27 V. It is also preferred that the electric charge be applied in the form of 2 to 100 pulses over a course of less than one hour. The electrically treated medium may be immediately used as a binding medium or it may be advantageous to wait from one second (preferably thirty seconds) to forty minutes to introduce the reagents into the medium to form the binding medium.

The electric charge can be applied to the binding medium while the first and second biopolymers are binding together, and/or after the first and second biopolymers have bonded together.

The binding medium of the invention is not particularly limited. Suitable binding media are discussed below with respect to the assay of the invention. The biopolymers can be free in solution or bonded to a solid support, such as a semipermeable membrane, microtiter plate, chip array, etc.

The binding medium can remain relatively stationary in a single vessel or move through a semipermeable apparatus, capillary or channel from one location to another.

The binding effects provided by the invention should not be confused with the effects reported by others. The present invention is not the electric denaturation taught by Stanley, supra. Thus, in embodiments wherein the first and second biopolymers form triplexes or quadruplexes, the first and second biopolymers need not be denatured.

Without wishing to be bound by any theories, we do not believe that the increased binding affinity or specificity is a result of electrophoretic migration of the biopolymers or electroporation of a membrane separating the biopolymers.

The method can be used to eliminate or reduce probe:probe binding when probe is in molar excess of nucleic acid target. In embodiments, it can also be useful to reduce or eliminate mismatch binding. It should be understood from this disclosure that the binding method of the invention has diagnostic, analytical, therapeutic, prophylactic and engineering applications.

The invention can also be employed in a variety of contexts, including the assay and apparatus discussed below.

Assay

The invention provides a rapid, sensitive, environmentally friendly, and safe system for assaying binding between a target and a probe, wherein the target comprises a nucleic acid sequence or a nucleic acid analogue sequence and the probe comprises a nucleic acid sequence or a nucleic acid analogue sequence. The system of the invention is also suitable for assaying binding between a target and a probe, wherein the target and/or the probe comprises an amino acid sequence. Thus, the invention is suitable for assaying binding of biopolymers.

Unlike certain prior art systems, the invention not only detects the presence of specific binding, but also provides qualitative and quantitative information regarding the nature of binding between a probe and target and like information about other binding, which the electrical treatment has diminished or prevented. Thus, in embodiments comprising nucleobase to nucleobase binding assays, the invention enables the practitioner to distinguish among a perfect match, a one base pair mismatch, a two base pair mismatch, a three base pair mismatch, a one base pair deletion, a two base pair deletion and a three base pair deletion.

Embodiments of the invention comprise calibrating a measured signal of any description (e.g., electric current and/or fluorescent intensity) for a first probe-target mixture against the same type of signal exhibited by other probes combined with the same target, wherein each of the other probes differs from the first probe by at least one base.

In certain embodiments, a low voltage is applied to the sample prior to or concurrent with measuring said signal. Generally, the voltage is selected such that it diminishes imperfectly matched hybridization but does not diminish perfectly matched hybridization. In certain preferred embodiments, the voltage is about 1 V to about 27 V. In certain embodiments, the signal is measured immediately after applying the electric treatment to the binding medium or up to forty minutes after application of the electrical treatment.

Calibration curves can be generated, wherein the magnitude of the measured signal (e.g., electric current and/or fluorescent intensity) is a function of the binding affinity between the target and probe when present in treated or untreated medium. As the binding affinity between the target and a plurality of different probes varies with electrical treatment of the medium and the number of mismatched bases in nucleobase-nucleobase assays, the nature of the mismatch (A-G vs. A-C vs. T-G vs. T-C, etc.), the location of the mismatch(es) within the hybridization complex, etc., the assay of the invention can be used to sequence the target.

The signal measured can be, e.g., electrical conductance. In such embodiments, the binding affinity between the probe and target is directly correlated with the magnitude of the signal. That is, the electrical conductance increases along with the extent of matching between the probe and target, preferably over a range inclusive of 0-2 mismatches and/or deletions, more preferably over a range inclusive of 0-3 mismatches and/or deletions.

In other embodiments, the signal measured can be the fluorescent intensity of a fluorophore included in the test sample. In such embodiments, the binding affinity between the probe and target can be directly or inversely correlated with the intensity, depending on whether the fluorophore signals hybridization through signal quenching or signal amplification. Thus, the fluorescent intensity generated by intercalating agents can be directly correlated with probe-target binding affinity, whereas the intensity of embodiments employing non-intercalating fluorophores covalently bound to the probe can be inversely correlated with probe-target binding affinity. The fluorescent intensity increases (or decreases for non-intercalators) along with the extent of matching between the probe and target, preferably over a range inclusive of 0-2 mismatches and/or deletions, more preferably over a range inclusive of 0-3 mismatches and/or deletions.

Although we have previously disclosed the advantages of fluorescent intensity assays for analyzing hybridization of nucleobase-containing sequences (see U.S. Pat. No. 6,403,313 to Daksis et al.) and the advantages of fluorescent intensity assays for analyzing peptide:nucleic acid binding (see U.S. Pat. No. 6,294,333) and peptide:peptide binding (see U.S. patent application Ser. No. 09/344,525, filed Jun. 25, 1999), the application of an electric field to the sample appears to increase the resolution of the assay, as shown in the Examples.

Moreover, in particularly preferred embodiments of the invention, the assay comprises measuring at least two signals of any description detected from the sample. The first signal is preferably fluorescent intensity and the second signal is preferably selected from several electrical conductance measurements (or vice versa).

In the preferred multiple measurement embodiments, the first signal can be the same as or different from the second signal. When the first and second signals measured are the same, the second signal can be calibrated against the first signal and/or against the same reference signal(s) used to calibrate the first signal. In addition, at least one condition-altering stimulus is preferably applied to the test sample after the first signal is measured and before the second signal is measured. The stimulus is preferably sufficient to measurably change binding, as indicated by at least one signal. In nucleobase-nucleobase binding assays of the invention, the stimulus is preferably sufficient to significantly affect imperfectly complementary hybridization between the probe and the target and insufficient to significantly affect perfectly complementary hybridization between the probe and the target.

In certain embodiments of the invention, at least one stimulus is applied once or a plurality of times. The stimulus can be continuously applied or non-continuously applied. The stimulus can be applied before, during and/or after the detection of signal detection.

Suitable stimuli can be, e.g., photonic radiation (such as laser light) and/or electronic. The signals detected can be, e.g., photonic and/or electronic as well.

For example, in a particularly preferred embodiment of the invention, the first signal measured is pre-electrification fluorescent intensity (i.e., intensity measured before a condition-altering voltage is applied to the test sample) and the second signal measured is post-electrification fluorescent intensity (i.e., intensity measured during or after the condition-altering voltage has been applied to the test sample).

The additional measurements in the foregoing embodiments increase the reliability of the assay and enable immediately retesting suspect results. Inconsistent results achieved by the at least two measurements will typically warrant retesting.

The invention enables quantifying the binding affinity between probe and target. Such information can be valuable for a variety of uses, including designing antisense drugs with optimized binding characteristics.

Unlike prior art methods, the assay of the invention is preferably homogeneous. The assay can be conducted without separating the probe-target complex from the free probe and target prior to detecting the magnitude of the measured signal. The assay does not require a gel separation step, thereby allowing a great increase in testing throughput. Quantitative analyses are simple and accurate. Consequently the binding assay saves a lot of time and expense, and can be easily automated. Furthermore, it enables binding variables such as buffer, pH, ionic concentration, temperature, incubation time, relative concentrations of probe and target sequences, intercalator concentration, length of target sequences, length of probe sequences, and possible cofactor requirements to be rapidly determined. The electrical treatment of medium can be tailored to complement binding variables otherwise selected by the practitioner or with which he or she is familiar.

The assay can be conducted in e.g., a solution within a well, on an impermeable surface, on a biochip, or in a channel or microchannel. In certain embodiments, it may be useful to employ as a sample support the Patterned Multi-Array Multi-Specific Surface PMAMS for electrochemiluminescent assays disclosed in published U.S. patent application Ser. No. 2001/0021534 A1 to Wohlstadter et al. and/or the sample support and associated sample handling devices disclosed in published U.S. patent application Ser. No. 2001/0051113 A1 to Juncosa et al.

Moreover, the inventive assay is preferably conducted without providing a signal quenching agent on the target or on the probe.

Preferred embodiments of the invention specifically detect triplex and/or quadruplex hybridization between the probe and the double-stranded target, thus obviating the need to denature the target. Duplex formation between a nucleic acid probe and target can also be detected. Triplex and quadruplex formation and/or stabilization is enhanced by the presence of an intercalating agent in the sample being tested. See, e.g., U.S. patent application Ser. No. 09/885,731, filed Jun. 20, 2001, and U.S. patent application Ser. No. 09/909,496, filed Jul. 20, 2001.

Suitable nucleobase-containing probes for use in the inventive assay include, e.g., ssDNA, RNA, PNA and other nucleic acid analogues having uncharged, partially-charged or positively charged backbones. Although antiparallel single strand probes are preferred in certain embodiments, single strand probes may also be parallel to the target sequence to be bound. Probe sequences having any length from 8 to 20 bases are preferred since this is the range within which the smallest unique DNA sequences of prokaryotes and eukaryotes are found. Probes of 12 to 18 bases are particularly preferred since this is the length of the smallest unique sequences in the human genome. In embodiments, probes of 5 to 30 bases are most preferred. However, a plurality of shorter probes can be used to detect a nucleotide sequence having a plurality of non-unique target sequences therein, which combine to uniquely identify the nucleotide sequence. The length of the probe can be selected to match the length of the target.

Suitable amino acid-containing probes can comprise a single amino acid, single amino acid analogue, a peptide-like analogue, peptidoid, peptidomimetic, peptide, dipeptide, tripeptide, polypeptide, protein or a multi-protein complex.

The invention does not require the use of radioactive probes, which are hazardous, tedious and time-consuming to use, and need to be constantly regenerated. Probes of the invention are preferably safe to use and stable for years. Accordingly, probes can be made or ordered in large quantities and stored.

In embodiments of the invention wherein the target comprises amino acids, the target preferably comprises a peptide sequence or a peptide-like analogue sequence, such as, e.g., a dipeptide, tripeptide, polypeptide, protein or a multi-protein complex. More preferably, the target is a protein having at least one receptor site for the probe.

In embodiments of the invention wherein the target comprises nucleobases, the targets are preferably 8 to 3.3×10⁹ base pairs long, and can be single or double-stranded sequences of nucleic acids and/or analogues thereof.

It is preferred that the probe and target be unlabeled, but in alternative embodiments, there is an intercalating agent covalently bound to the probe. In such embodiments, the intercalating agent is preferably bound to the probe at either end.

In other embodiments, the intercalating agent is not covalently bound to the probe, although it can insert itself into the probe-target complex during the assay.

Preferred intercalating agents for use in the invention include, e.g., YOYO-1, TOTO-1, ethidium bromide, ethidium homodimer-1, ethidium homodimer-2 and acridine. In general, the intercalating agent is a moiety that is able to interact with strands of a duplex, triplex and/or a quadruplex nucleic acid complex. In preferred embodiments, the intercalating agent (or a component thereof) is essentially non-fluorescent in the absence of nucleic acids and fluoresces when interacting with the nucleic acids and is excited by radiation of an appropriate wavelength, exhibiting a 100-fold to 10,000-fold enhancement of fluorescence when intercalated within a duplex or triplex nucleic acid complex.

In alternative embodiments, the intercalating agent may exhibit a shift in fluorescent wavelength upon intercalation and excitation by radiation of an appropriate wavelength. The exact fluorescent wavelength may depend on the structure of the nucleic acid that it interacts with, for example, DNA vs. RNA, duplex vs. triplex, etc.

The excitation wavelength is selected (by routine experimentation and/or conventional knowledge) to correspond to this excitation maximum for the fluorophore being used, and is preferably 200 to 1000 nm. Intercalating agents are preferably selected to have an emission wavelength of 200 to 1000 nm. In preferred embodiments, an argon ion laser is used to irradiate the fluorophore with light having a wavelength in a range of 400 to 540 nm, and fluorescent emission is detected in a range of 500 to 750 nm.

The assay of the invention can be performed over a wide variety of temperatures, such as, e.g., from 5 to 85° C. Certain prior art assays require elevated temperatures, adding cost and delay to the assay. On the other hand, the invention can be conducted at room temperature or below (e.g., at a temperature below 25° C.).

The inventive assay is extremely sensitive, thereby obviating the need to conduct PCR amplification of the target. For example, in at least the fluorescent intensity embodiments, it is possible to assay a test sample having a volume of about 20 microliters, which contains about 10 femtomoles of target and about 10 femtomoles of probe. Embodiments of the invention are sensitive enough to assay genomic targets or targets at a concentration of 5×10⁻⁹ M, preferably at a concentration of not more than 5×10⁻¹⁰ M. Embodiments of the invention are sensitive enough to employ probes at a concentration of 5×10⁻⁹ M, preferably at a concentration of not more than 5×10⁻¹⁰ M.

Conductivity measurements can distinguish samples having as little as about 1 pmole of probe and 1 pmole of target in 40 microliters of medium. Decreasing the sample volume will permit the use of even smaller amounts of probe and target.

It should go without saying that the foregoing values are not intended to suggest that the method cannot detect higher concentrations.

A wide range of intercalator concentrations is tolerated at each concentration of probe and target tested. For example, when 5×10⁻¹⁰ M probe and 5×10⁻¹⁰ M target are hybridized, the optimal concentration of the intercalator YOYO-1 ranges from 25 nM to 2.5 nM. At a 5×10⁻⁸ M concentration of both probe and target, the preferred YOYO-1 concentration range is 1000 nM to 100 nM.

The assay is sufficiently sensitive to distinguish a one base-pair mismatched probe-target complex from a two base-pair mismatched probe-target complex, and preferably a two base-pair mismatched probe-target complex from a three base-pair mismatched probe-target complex. Of course, the assay is sufficiently sensitive to distinguish a perfectly matched probe-target complex from any of the above mismatched complexes.

The binding medium can be any conventional medium known to be suitable for preserving nucleotides and/or proteins. See, e.g., Sambrook et al., “Molecular Cloning: A Lab Manual,” Vol. 2 (1989). For example, the liquid medium can comprise nucleotides, water, buffers and standard salt concentrations.

Hybridization between complementary bases occurs under a wide variety of conditions having variations in temperature, salt concentration, electrostatic strength, and buffer composition. Examples of these conditions and methods for applying them are known in the art.

It is preferred that hybridization complexes be formed at a temperature of about 5° C. to about 25° C. for about 1 minute to about 5 minutes. Longer reaction times are not required, but incubation for several hours may not adversely affect the hybridization complexes.

It is possible (although unnecessary, particularly for embodiments containing an intercalating agent) to facilitate hybridization in solution by using certain reagents. Preferred examples of these reagents include single stranded binding proteins such as Rec A protein, T4 gene 32 protein, E. coli single stranded binding protein, major or minor nucleic acid groove binding proteins, divalent ions, polyvalent ions, viologen and intercalating substances such as ethidium bromide, actinomycin D, psoralen, and angelicin. Such facilitating reagents may prove useful in extreme operating conditions, for example, under abnormal pH levels or extremely high temperatures.

The inventive assay can be used to, e.g., identify accessible regions in folded nucleotide sequences, to determine the number of mismatched base pairs in a hybridization complex, and to map genomes.

In embodiments wherein fluorescent intensity is detected using an intercalating agent, intensity increases with increasing binding affinity between the probe and target. In embodiments wherein fluorescent intensity is detected using a non-intercalating, quenching fluorophore, intensity decreases as binding affinity increases between the probe and target. Regardless of whether the fluorophore intercalates or not, the instant method does not require the measurement of the polarization of fluorescence, unlike fluorescent anisotropy methods, although the measurement of polarization of fluorescence may be one of the signals detected from the sample, as referred to above.

Apparatus

A preferred apparatus for performing the binding method and the assay of the invention includes a light source, an electric source, sample handling means, a photon detector, an electron detector and a data analysis device.

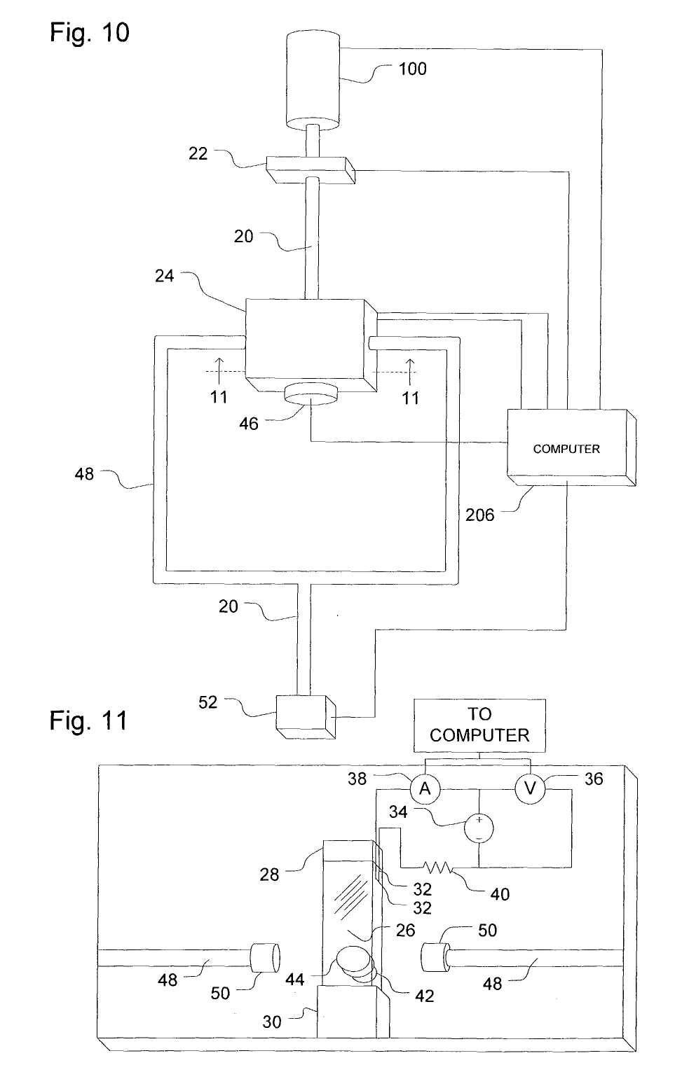

Referring to FIGS. 10 and 11, a preferred apparatus of the invention includes a laser as light source 100. Non-coherent (or polychromatic) light generation devices are also suitable for use in the invention. Light source 100 is preferably an argon laser that generates a beam having a wavelength of about 488 nm, which in some embodiments may be a model 2017 or model 161C manufactured by Spectra-Physics or a model 170B manufactured by Omnichrome (now Melles Griot). Other lasers, such as diode lasers, helium lasers, dye lasers, titanium sapphire lasers, Nd:YAG lasers or others can also be employed.

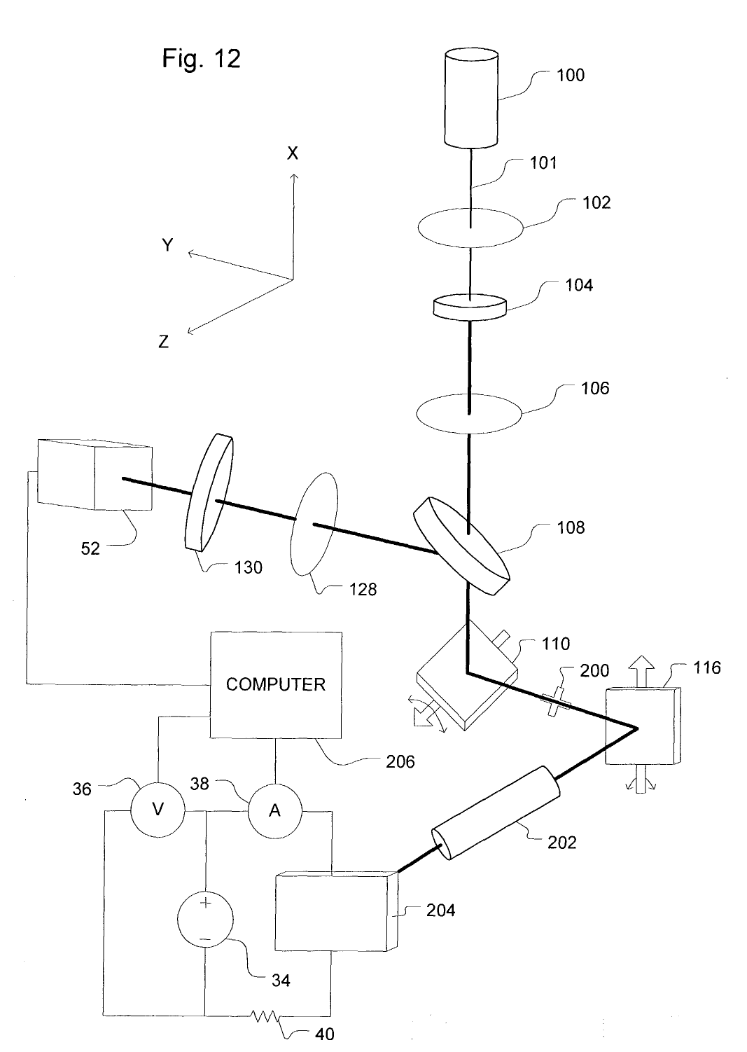

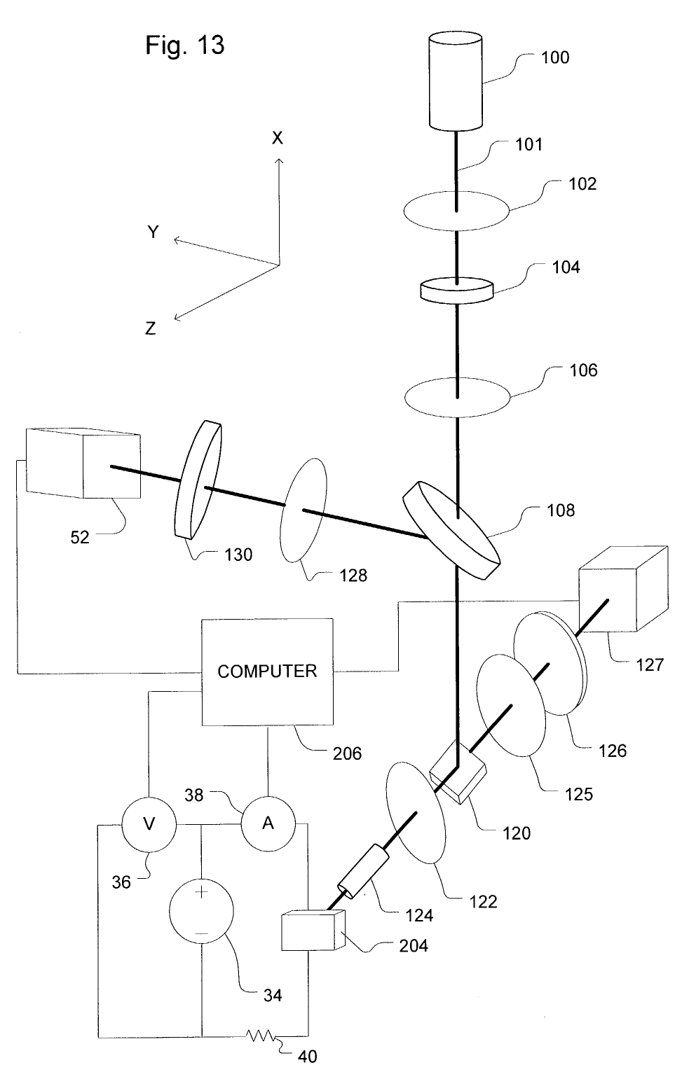

Light source 100 emits a beam (not shown in FIG. 10), which is conveyed through optical fiber 20 past shutter 22 and into sample chamber 24. Light source 100 and shutter 22 are preferably controlled by computer 206 (e.g., a PENTIUM 4 based PC) to provide a desired amount of radiation to sample 26 within sample chamber 24. When taking measurements with the apparatus, light emission data collected when the shutter is being opened or closed should be discarded. For example, the data acquisition can be started about 25 milliseconds after opening the shutter and stopped about 25 milliseconds before closing the shutter. A shutter is optionally provided in the embodiments shown in FIGS. 12 and 13.

Sample 26 is placed within sample chamber 24 by, e.g., opening a lid (not shown) of sample chamber 24 and placing sample container 28 within holder 30. Electrodes 32 are placed within sample 26 for purposes of conducting electricity into sample 26 and/or measuring electrical characteristics of sample 26. Electrodes 32 are a part of an electric circuit including voltage source 34, voltmeter 36, ammeter 38 and resistor 40 provided for the purpose of performing the measurements of the electrical characteristics of the sample 26. Voltage source 34 preferably provides direct current, and can be controlled and monitored via computer 206 using voltmeter 36 and ammeter 38. The monitored values, along with the known value of resistor 40, are used by computer 206 to determine the electrical characteristics of sample 26.

After placing sample 26 in sample chamber 24 and immersing electrodes 32 in the sample, the lid of the sample chamber is replaced to minimize the deleterious effects of extraneous light interfering with the method of the invention. Sample chamber 24 is preferably constructed of opaque materials (e.g., black or blackened metal) in a manner intended to seal out extraneous (ambient) light, unless the sample chamber along with other parts of the apparatus are housed within a light-tight housing.

Although the embodiment depicted in FIGS. 10 and 11 is suitable for manual positioning of the sample, it is also within the scope of the invention to provide automated sample handling means. For example, holder 30 and sample container 28 can be provided on a moving substrate, such as a conveyor belt or rotating platter, or manipulated by a robotic arm to convey sample 26 into and out of sample chamber 24.

Likewise, sample container 28, which is shown as a cuvette (preferably quartz) in FIG. 11, can be provided in a form more conducive to automated, high-throughput sample analysis, such as, e.g., microtiter plates and other sample arrays. In such embodiments, it may be more practical to provide electrodes as part of the container, which would complete the electric circuit when the container is properly positioned within the sample chamber. In any case, it is preferred that sample container 28 be constructed from materials that are transparent or translucent at the wavelength(s) of excitation and emission.

After loading sample 26 in sample chamber 24, light is irradiated from optical fiber input 42 through input filter 44 and into sample 26. Input filter 44 is selected to minimize the amount of light passing to the sample (or even entering sample chamber 24) that has a wavelength other than the excitation wavelength for the fluorophore within the sample. Input filter 44 can be a narrow pass filter, a low pass filter or a high pass filter. For example, when YOYO-1 is used as the marking agent in the sample, input filter 44 can comprise a low pass filter that prevents the emission of light from optical fiber input 42 having a wavelength greater than about 500 nm. Since YOYO-1 has a maximum fluorescent intensity emission at about 536 nm, the low pass filter prevents any light from optical fiber input 42 of similar wavelength from erroneously inflating the detected amount of light emitted by YOYO-1. Of course, a narrow pass filter or a combination of a high pass filter and a low pass filter can be used to pinpoint a wavelength (or range of wavelengths) of light to be emitted from optical fiber input 42, and is particularly useful with embodiments employing a non-coherent light source.

Referring to FIG. 10, a sensor 46 is optionally used to monitor the power of the beam as applied in the sample chamber 24. Sensor 46 is placed opposite optical fiber input 42 such that light passing through sample 26 strikes the sensor. The output from sensor 46 is routed to computer 206 for analysis and reporting. Sensor preferably comprises a photoelectric cell, such as a photodiode, phototransistor or the like. Sensor 46 enables the operator of the apparatus to confirm the accuracy of the power settings reported by light source 100 to computer 206.

Fluorescent radiation emitted from sample 26 is collected by optical fiber outputs 48, which are preferably mounted perpendicular to optical fiber input 42 to maximize the amount of fluorescent radiation (which is emitted at an angle perpendicular to the axis of incident exciting radiation from optical fiber input 42) collected from the sample. In the embodiment of FIGS. 10 and 11, two optical fiber outputs 48 are shown, but the invention encompasses the use of more or less of these outputs.

The radiation conveyed through optical fiber outputs 48 is filtered by output filters 50 to minimize the amount of extraneous light detected by the apparatus. Output filters 50 can be independently selected from the group consisting of narrow pass filters, low pass filters and high pass filters. For example, when YOYO-1 is used as the marking agent in the sample, output filters 50 can comprise a high pass filter that prevents the passage of light from sample chamber 24 having a wavelength less than about 500 nm. Since YOYO-1 has a maximum fluorescent intensity emission at about 536 nm, the high pass filter prevents excitation light (of about 488 nm) from erroneously inflating the detected amount of light emitted by YOYO-1. Of course, a narrow pass filter or a combination of a high pass filter and a low pass filter can be used to pinpoint a wavelength (or range of wavelengths) of light to be passed through optical fiber outputs 48.

The radiation collected by optical fiber outputs 48 is conveyed to detector 52, which reports to computer 206. Detector 52 preferably comprises a CCD.

FIG. 12 shows an alternative embodiment of the apparatus of the invention, which is particularly suitable for scanning an array of samples on a substrate. In FIG. 12, the incoming beam 101 from light source 100 passes through a spatial filter and beam expander (comprising lens 102, pinhole 104 and lens 106), and is expanded to match the diameter of the entrance pupil 200 of laser scan lens 202. The spatial filter and beam expander is optionally provided in front of light source 100 to improve the Gaussian profile of beam 101. Lens 102 and 106 can be, for example, 0.5 inch (1.27 cm) diameter 50 mm focal length anti-reflection coated piano convex glass lens or equivalent. Both lenses are preferably configured such that both their back focal planes coincide with pinhole 104. Pinhole 104 can have a wide range of aperture diameters, such as, e.g., 1-1000 μm, and preferably about 30 μm.

Scanning mirrors 110 and 116 deflect the beam in a raster scan, and rotate about axes that are perpendicular to each other and are placed close together, on either side of the entrance pupil of the laser scan lens. Laser scan lens 202 focuses the beam to a spot on sample array 204 (e.g., microtiter plate), and reflected light is collected by laser scan lens 202, descanned by scanning mirrors 116 and 110, and partially reflected by beam splitter 108 into a confocal detection arm comprised of lens 128, pinhole 130 and detector 52. In embodiments, pinhole 130 can be removed to provide a non-confocal imaging system. Light reflected back from the focused spot on the sample passes through pinhole 130 and is detected, but light from any other point in the sample runs into the edges of the pinhole and is not detected. The scan mirrors are computer-controlled to raster the focused spot across the sample.

Beamsplitter 108 is preferably a dichroic beamsplitter, which reflects the longer-wavelength fluorescence (or shorter-wavelength fluorescence in the case of up-converting labels, which emit radiation at shorter wavelengths than the excitation radiation—see, e.g., U.S. Pat. No. 5,674,698 to Zarling et al.) returning from the specimen into the confocal detection arm, while allowing reflected light, at the excitation wavelength, to pass through. In certain embodiments, beam splitter 108 can be, for example, a non-polarizing 50% beam splitter cube made by Melles Griot model number 03BSC007 or equivalent.

Computer 206 is connected to the detector 52 to receive, analyze, store and/or display a signal from the detector 52. Laser scan lenses are not usually used in imaging systems, and a beam of light will be collected by the lens that is wider than the incoming laser beam, but only the component of this beam that is parallel to and concentric with the incoming laser beam will pass through the pinhole and be detected. Thus, this is a true confocal imaging system, and will have optical image slicing properties similar to those of a confocal scanning laser microscope, except applied to much larger samples.

In certain embodiments, a stop with the same diameter as entrance pupil 200 of laser scan lens 202 can be placed at the entrance pupil position (just to the left of scanning mirror 116 in FIG. 12) if required, to reduce the out-of-focus part of the returning beam traveling back toward the confocal detector.

In certain embodiments wherein scanning mirror 116 has beam splitting properties, a detector is placed behind the mirror to detect non-confocal light. The beam splitting mirror is preferably dichroic and reflects light at the excitation wavelength returning from the specimen, while allowing the longer-wavelength fluorescence (or shorter-wavelength fluorescence in the case of up-converting labels) to pass through. Suitable dichroic mirrors include, e.g., a LWP-45°S-488R/520T-1025 made by CVI Laser Corp. or equivalent.

Alternatively, a beam splitter between sample array 204 and laser scan lens 202 can be used to divert a portion of light emitted from the sample array through a condenser lens to a detector. The beam splitter reflects the longer-wavelength fluorescence (or shorter-wavelength fluorescence in the case of up-converting labels) returning from the specimen into the condenser lens and detector, while allowing reflected light, at the excitation wavelength, to pass through.

FIG. 13 shows an alternative embodiment of the apparatus of the invention, which is particularly suitable for scanning an array of samples on a substrate.

Sample array 204 is preferably transparent to a wide spectrum of light. In some embodiments, sample array 204 is made of a conventional microscope glass slide or cover slip. It is preferable that the sample array be as thin as possible while still providing adequate physical support. Preferably, the sample array is less than about 1 mm thick, more preferably less than 0.5 mm thick. Typically, the sample array is a microscope glass slide of about 0.7 mm or 700 μm thick. In alternative embodiments, the sample array may be made of quartz or silica.

Sample array 204 can optionally be mounted on a flow cell as taught by U.S. Pat. No. 6,141,096 to Stern et al.

Light source 100 generates beam 101 to excite fluorescent targets in the flow cell. The laser is directed at sample array 204 through an optical train comprised of various optical elements which will be described below in detail to the extent that such elements differ from the embodiment of FIG. 12.

After passing through beam splitter 108, the excitation light is reflected by dichroic mirror 120. In certain embodiments, dichroic mirror 120 passes light having a wavelength greater than about 520 nm, but reflects light having a wavelength of about 488 nm. Consequently, the 488 nm light from the laser is reflected by dichroic mirror 120 toward optical lens 122. In certain embodiments, optical lens 122 is a 0.5 inch (1.27 cm) diameter—50 mm focal length anti-reflection coated plano-concave glass lens made by Newport or equivalent. The light then passes through a microscope objective 124 to sample array 204 for magnification of the sample image. Microscope objective 124, in some embodiments, may be a 10×0.3 NA microscope objective, but other magnifications could also be used. In a preferred embodiment, the distance between lens 122 and microscope objective 124 is about 100 mm.

Microscope objective 124 focuses the light on samples in sample array 204. Preferably, the microscope objective produces a spot about 2 μm in diameter in its focal plane. The optical train described in the above embodiments produces a 2 μm diameter focal spot when used with a laser which generates a beam diameter of 1.4 mm, such as the Spectra-Physics model 2017.

In alternative embodiments, the 2 μm spot may be easily obtained when other types of light sources with different beam diameters are used. Since the diameter of the focal spot is inversely proportional to the diameter of the collimated beam produced by lens 106, the desired spot size may be achieved by varying the focal lengths of the spatial filter. Alternatively, a beam expander may be used to expand or compress the beam from the light source to obtain the desired spot size. For example, with a model 161C, which generates a beam diameter of 0.7 mm, a 2 μm diameter focal spot may be achieved if the ratio of the lens in the spatial filter is 1:2 instead of 1:1. Thus, by varying the focal lengths of the lenses in the spatial filter and/or using a beam expander, the appropriate excitation spot size may be achieved from various beam diameters.

In a preferred embodiment, the 2 μm spot has a power of 50 μW. Depending on the light source used, a variable neutral density filter can be inserted between the laser 100 and the optical train to attenuate the power of the laser to the desired power level.

Fluorescent emissions are collected by the microscope objective 124 and passed to optical lens 122. Optical lens 122 collimates the fluorescence and passes it to dichroic mirror 120. In practice, light collected by microscope objective contains both fluorescence emitted by the fluorescein and 488 nm laser light reflected from the sample array 204. The laser component reflected from the sample array is reflected by dichroic mirror 120 back to beam splitter 108. Beam splitter 108 directs the laser component through a lens 128. The lens, in some embodiments, can be 0.5 inch (1.27 cm) diameter—50 mm focal length anti-reflection coated plano convex glass lens made by Newport, but equivalent thereof may be used. Lens 128 focuses the laser component to detector 52. Preferably, a confocal pinhole 130 is located between lens 128 and detector 52. Pinhole 130 transmits substantially only the reflected light originating from the focal plane of the microscope to detector 52, while reflected light originating from out-of-focus planes are blocked. In certain embodiments, pinhole 171 has an aperture of about 50 μm.

Detector 52 can be, e.g., a photodiode that generates a voltage corresponding to the intensity of the detected light. The photodiode can be, e.g., a 13 DSI007 made by Melles Griot or equivalent, or other light detection devices, such as photomultiplier tube or avalanche photodiode may be used. Output from detector 52 is used by computer 206 to focus the laser at a point within a sample on sample array 204.

As for the fluorescent component emitted from sample array 204, most of it will pass through the dichroic mirror 120. The fluoresced light is then focused by a lens 125 to detector 127 (e.g., a photomultiplier tube) for detecting the number of photons present therein. Lens 125, in a preferred embodiment, is a 0.5 inch (1.27 cm) diameter—50 mm focal length anti-reflection coated plano convex glass lens made by Newport, but equivalent lens may be used. A pinhole 126 is preferably located between lens 125 and detector 127. Pinhole 126 transmits fluorescence originating from the focal plane and filters out light originating from other planes, such as from the glass or reagent. Accordingly, the signal-to-noise ratio of the fluoresced light is increased.

Additionally, a filter (not shown) is preferably located between detector 127 and pinhole 126 to filter out light having a wavelength other than the wavelength(s) of fluorescent emission. The filter further ensures that detector 127 detects substantially only fluoresced light.

In certain embodiments, detector 127 is a Hamamatsu R4457P photomultiplier tube with Hamamatsu C3866 preamplifier/discriminator. The photomultiplier tube generates approximately a 2 mV pulse for each photon detected. Each of these 2 mV pulses are converted to a TTL pulse by the preamplifier/discriminator. The TTL pulses, each one corresponding to a photon detected by the photomultiplier tube, are then collected by a data acquisition board, such as a National Instruments “Lab-PC+” or equivalent, which typically contains an Intel 8254 or equivalent counter/timer chip. The data represent the photon count as a function of sample array position.

After data are collected from a region (i.e., sample) of the sample array, sample array 204 is moved so that light can be directed at a different region on the sample array. Movement of sample array can be accomplished by a variety of means, including but not limited to, a conveyor belt, a rotating disk, an x-y-z table, or the like. The process is repeated until all samples on the sample array have been scanned. By counting the number of photons generated in a given area in response to the excitation light, it is possible to determine where fluorescently marked molecules are located on the sample array. Consequently, it is possible to determine which of the probes within a matrix of probes is complementary to a fluorescently marked target.

According to preferred embodiments, the intensity and duration of the light applied to the sample array is controlled by computer 206. By varying the laser power and scan stage rate, the signal-to-noise ratio may be improved by maximizing fluorescence emissions. As a result, the present invention can detect the presence or absence of a target on a probe as well as determine the relative binding affinity of probes and targets.

As in the embodiments depicted in FIGS. 10-12, an electric circuit comprising electrodes 32, voltage source 34, voltmeter 36, ammeter 38 and resistor 40, is provided in electrical communication with the samples.

The invehtion will be illustrated in more detail with reference to the following Examples, but it should be understood that the present invention is not deemed to be limited thereto.

### EXAMPLES

### Example 1

Sense and antisense 15-mer and 50-mer ssDNA sequences, possessing a 52% GC content, were synthesized on a DNA synthesizer (Expedite 8909, PerSeptive Biosystems) and purified by HPLC. ssDNA oligonucleotides were dissolved in ddH₂O and diluted to a concentration of 1 pmole/μl. Equimolar amounts of complementary oligonucleotides were also denatured at 95° C. for 10 minutes and allowed to anneal gradually in the presence of 10 mM Tris, pH 7.5, 1 mM EDTA and 100 mM NaCl, as the temperature cooled to 21° C. over 1.5 hours. dsDNA oligonucleotides were diluted in ddH₂O to a concentration of 1 pmole/μl.

SEQ ID NO:1 and SEQ ID NO:2 were 15-mer ssDNAs with an attached fluorescein moiety at the 5′ position, and were antiparallel complementary to each other.

Sequence for SEQ ID NO:1: 5′-Flu-CTG TCA TCT CTG GTG-3′

Sequence for SEQ ID NO:2: 5′-Flu-CAC CAG AGA TGA CAG-3′

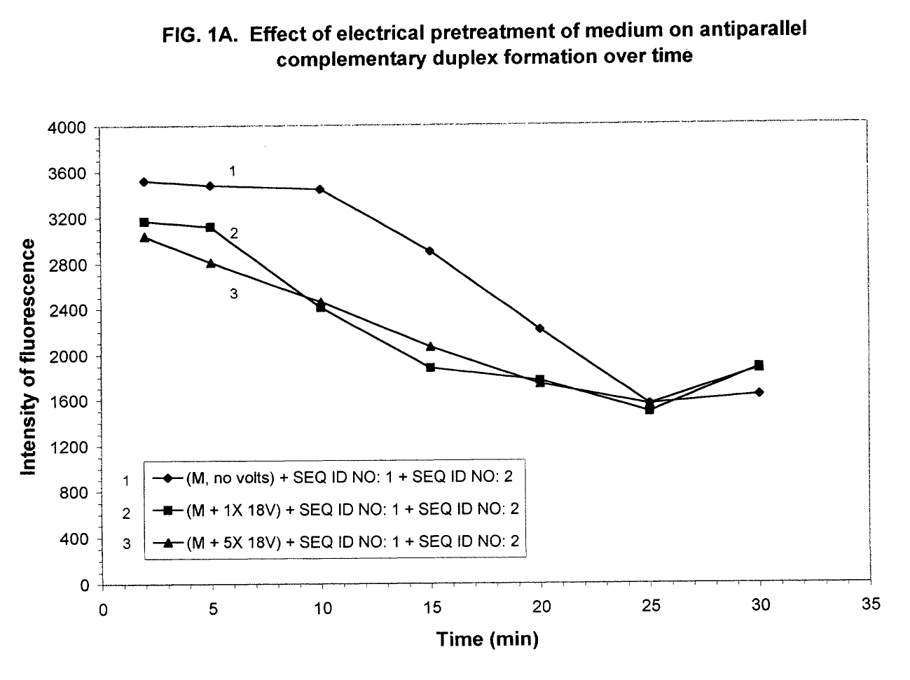

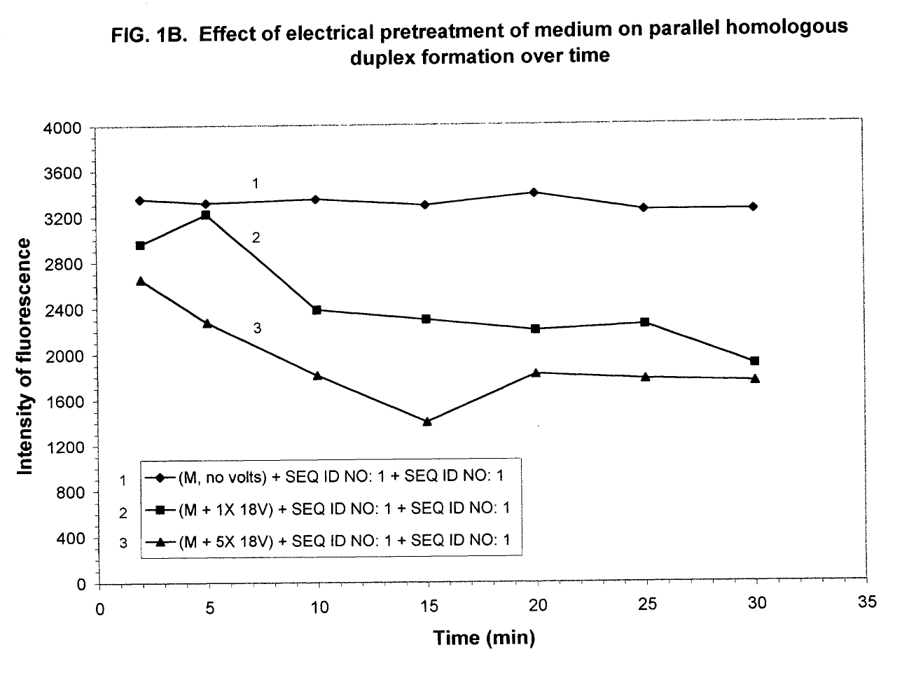

Aliquots of test medium comprising 10 mM Tris, pH 7.5 and 50 mM NaCl either remained untreated or were electrically pretreated prior to addition of DNA. The test medium was electrically pretreated by means of two platinum/iridium electrodes 2 mm apart. Three mm of each electrode was submerged in the liquid in a 1.5 ml microcentrifuge tube containing 76 μl of test medium. Either one or five applications of 18 volts of DC current were applied for 500 msec periodically to the test medium, with a 30 second interval between each 18V application. Two pmoles of SEQ ID NO:1 and two pmoles of SEQ ID NO:2 (FIG. 1A) or four pmoles of SEQ ID NO:1 (FIG. 1B) were added immediately to the untreated or electrically pretreated test medium to give a final volume of 80 μl. Identical samples, incubated individually for up to 30 min after addition of DNA, were placed into a 3 mm quartz cuvette, irradiated for 250 msec with an argon ion laser beam having a wavelength of 488 nm and monitored for fluorescent emission. The first fluorescent measurement was taken two minutes after addition of the DNA. The maximum fluorescent intensities occurred at a wavelength of 525 nm, the emission wavelength for fluorescein. The intensity of fluorescence was plotted as a function of incubation time for each sample analyzed.

In the untreated test medium antiparallel complementary ssDNA:ssDNA duplex formation between SEQ ID NO:1 and SEQ ID NO:2 started after 10 minutes of incubation and reached a maximum after 25 minutes of incubation (FIG. 1A). A 55.6% decrease in fluorescence was observed when comparing values acquired at 2 minutes and 25 minutes, indicative of duplex formation in the untreated medium. Antiparallel complementary duplex formation between SEQ ID NO:1 and SEQ ID NO:2 was more rapid in the electrically pretreated medium, where a single 18V pulse resulted in a 10.0% decrease in fluorescent intensity after a 2 minute incubation. When five 18V pulses were applied to the medium, a 13.6% decrease in fluorescence after 2 minutes was observed compared to that obtained in the non-electrified sample after 2 minutes (FIG. 1A). After 25 minutes of incubation similarly quenched fluorescent signals indicated that similar levels of antiparallel complementary duplex formation had been achieved in the pre-electrified and non-electrified media. At that timepoint a 57.6% and 55.7% decrease in fluorescence was observed following a one 18V pulse and five 18V pulses, respectively, when compared to that obtained at the 2 minute timepoint in the non-electrified sample (FIG. 1A).

No parallel homologous ssDNA:ssDNA duplex formation between strands of SEQ ID NO:1 was observed in the untreated medium during 30 minutes of incubation (FIG. 1B). In contrast, pre-electrification of the test medium noticeably promoted parallel homologous duplex formation, resulting in a 11.7% and 21.0% decrease in fluorescence at the 2 minute timepoint, and a 41.3% and 46.0% decrease after 30 minutes for the one 18V pulse and five 18V pulse pretreated samples, respectively (FIG. 1B).

500 msec applications of 9V of DC voltage similarly applied to the test medium before introduction of DNA, also promoted both antiparallel complementary and parallel homologous duplex formation. The enhancement of duplex formation was less when 9V pre-treatments were applied than when 18V pre-treatments were applied (data not shown). All incubations in this Example and in all other Examples proceeded at room temperature.

### Example 2

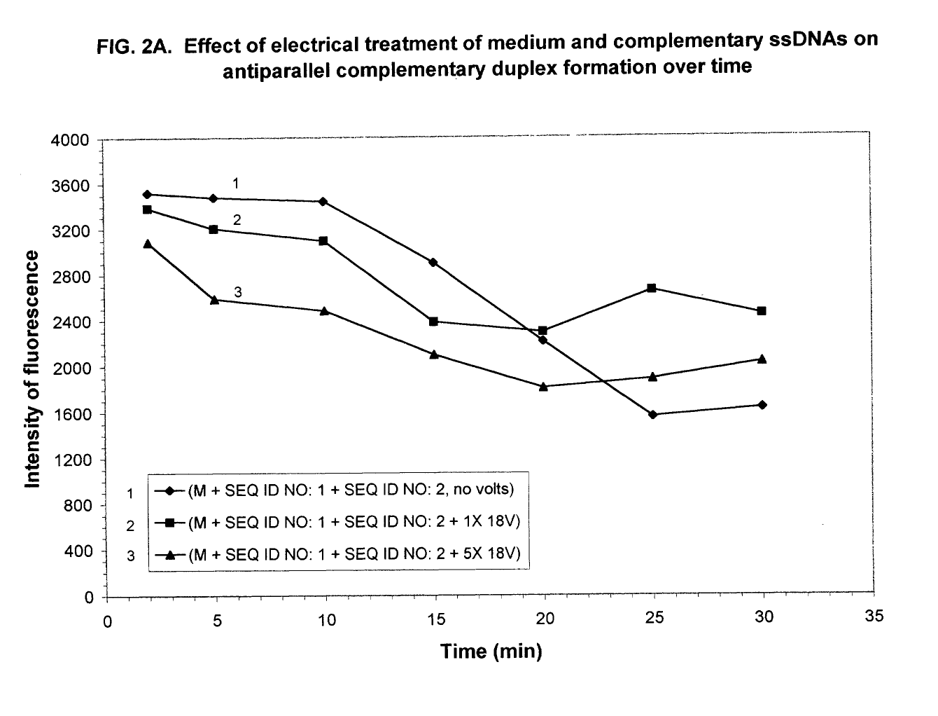

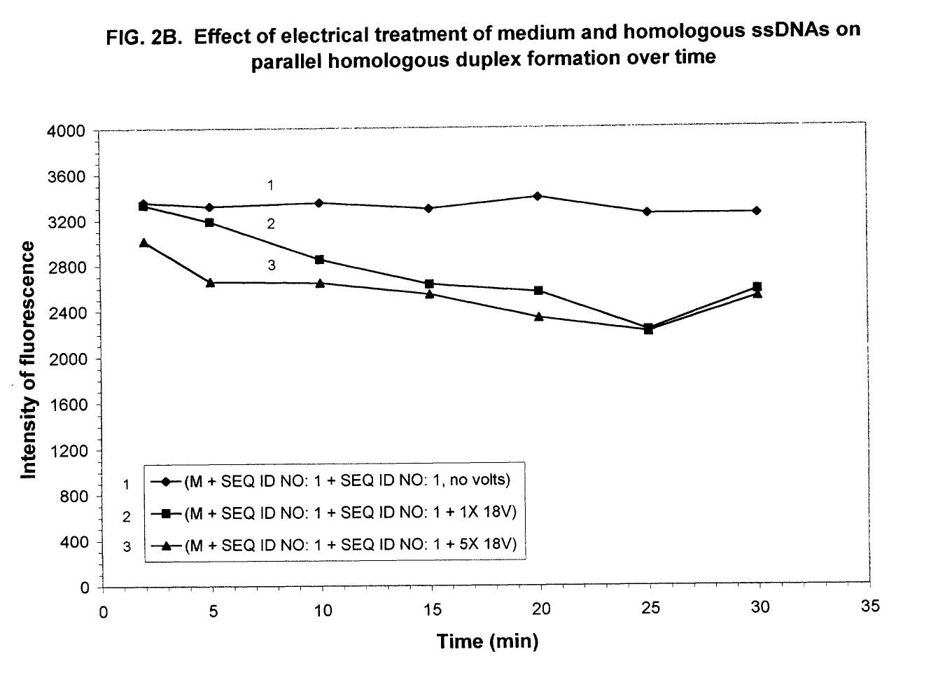

The effect of electrical pretreatment of medium on antiparallel complementary duplex binding or parallel homologous duplex binding was examined in Example 1. Example 2 demonstrates the effect of electrical treatment of medium containing complementary ssDNAs or homolog us ssDNAs on their duplex binding. Either one or five 500 msec applications of 18V of DC voltage were promptly applied to the test media to which had been added either two pmoles of SEQ ID NO:1 and two pmoles of SEQ ID NO:2 (FIG. 2A) or four pmoles of SEQ ID NO:1 (FIG. 2B) as described in Example 1. Identical samples were incubated individually for up to 30 min after electrification, were placed into a 3 mm quartz cuvette, irradiated for 250 msec with an argon ion laser beam having a wavelength of 488 nm and monitored for fluorescent emission. The first fluorescent measurement was taken two minutes after electrification of the sample. The intensity of fluorescence was plotted as a function of incubation time for each sample analyzed.

Antiparallel complementary duplex formation was initiated slightly more rapidly after one 18V pulse or substantially more rapidly after five 18V pulses of the test media containing SEQ ID NO:1 and SEQ ID NO:2, resulting in a 3.8% and 12.4% decrease in fluorescence, respectively, at the two minute timepoint, compared to the level of fluorescence observed in the non-electrified sample at that timepoint (FIG. 2A). The enhancement effect of electrical treatment lasted for a slightly shorter time, 20 minutes as compared to 25 minutes, when compared to Example 1. The level of fluorescence had decreased 37.0%, 34.7% and 48.4% after 20 minutes in the non-electrified sample, the once treated sample and the five times treated sample, respectively, compared to that achieved by the untreated sample at the 2 minute timepoint (FIG. 2A). The rate of duplex formation in the one 18V treated samples was slower when the ssDNAs were present during electrification than when the ssDNAs were added after electrical pretreatment of the medium alone (compare FIG. 2A and FIG. 1A). After 20 minutes of incubation, electrification of the test media containing SEQ ID NO:1 and SEQ ID NO:2 seemed to suppress antiparallel complementary duplex formation.

Electrical treatment of test medium containing 2 pmoles of SEQ ID NO:1, followed by subsequent addition of 2 pmoles of SEQ ID NO:2 gave similar results to that obtained when both SEQ ID NO:1 and SEQ ID NO:2 were present during the application of voltage (data not shown).

Electrification of the test medium containing SEQ ID NO:1 also increased parallel homologous duplex formation over the 30 minute incubation period, but the enhancement was substantially less when compared to that observed when the test medium alone was pre-electrified (compare FIG. 2B and FIG. 1B). The once treated samples and the five times treated samples showed a 0.7% and 10.1% decrease in fluorescence after 2 minutes, and a 23.2% and 24.9% decrease after 30 minutes, respectively, compared to that observed in the non-electrified sample at the 2 minute timepoint (FIG. 2B).

Electrical treatment of test medium containing 2 pmoles of SEQ ID NO:1, followed by subsequent addition of 2 pmoles of SEQ ID NO:1 gave similar results to that obtained when 4 pmoles of SEQ ID NO:1 were present in the medium during the application of voltage (data not shown).

500 msec applications of 9V of DC voltage applied to the test medium containing complementary ssDNAs or homologous ssDNAs also promoted both antiparallel complementary and parallel homologous duplex formation, however the enhancement of duplex formation was less than with the 18V treatments (data not shown).

Collectively these results suggest that antiparallel complementary duplex binding and parallel homologous duplex binding occurs at a greater rate when the medium alone has been electrically pretreated followed by the subsequent addition of the ssDNA strands, than when the medium containing the ssDNA strands are similarly treated electrically. It is important to note that the effect of electrical treatment or pretreatment occurs to some extent if only a portion of the final test volume has been so treated.

### Example 3

Example 3 compares the efficiency of formation of dsDNA duplexes in the presence of the DNA intercalator YOYO-1, when wild-type or mutant ssDNA target sequences are reacted with Watson-Crick complementary antiparallel ssDNA probes or with parallel homologous ssDNA probes in untreated test medium or following electrical pretreatment of the test medium.

Sequence for the sense strand of the wild-type 15-mer DNA probe (SEQ ID NO:3): 5′-CTG TCA TCT CTG GTG-3′

Sequence for the antisense strand of the wild-type 15-mer DNA probe (SEQ ID NO:3): 5′-CAC CAG AGA TGA CAG-3′

SEQ ID NO:4 was a 50-mer dsDNA target sequence. A central region of SEQ ID NO:4 was identical in sequence to that of SEQ ID NO:3.

Sequence for the sense strand of the wild-type target DNA (SEQ ID NO:4): 5′-GAG CAC CAT GAC AGA CAC TGT CAT CTC TGG TGT GTC CTA CGA TGA CTC TG-3′

Sequence for the antisense strand of the wild-type target DNA (SEQ ID NO:4): 5′-CAG AGT CAT CGT AGG ACA CAC CAG AGA TGA CAG TGT CTG TCA TGG TGC TC-3′

SEQ ID NO:5 was a 50-mer mutant dsDNA target sequence identical to SEQ ID NO:4, except for a one base pair mutation (underlined), at which the sequence CTC was changed to CTT.

Sequence for the sense strand of mutant SEQ ID NO:5: 5′-GAG CAC CAT GAC AGA CAC TGT CAT CTT TGG TGT GTC CTA CGA TGA CTC TG-3′

Sequence for the antisense strand of mutant SEQ ID NO:5: 5′-CAG AGT CAT CGT AGG ACA CAC CAA AGA TGA CAG TGT CTG TCA TGG TGC TC-3′

500 msec pulses of nine volts of DC current were applied to the test medium consisting of 0.7×TBE. When the total number of pulses was less than or equal to 30, a 30 second interval passed between each voltage application. When the total number of pulses was greater than 30, the interval between each 9V pulse was 10 seconds. A 56 μl volume of test medium was electrified by means described in Example 1 above. Immediately after the final pulse of DC current, two pmoles of the 15-mer ssDNA probe (sense strand of SEQ ID NO:3), two pmoles of the 50-mer ssDNA target and YOYO-1 were added to the untreated and electrically pretreated test media to give a final volume of 80 μl. The final buffer concentration was 0.5×TBE, and the final YOYO-1 concentration was 500 nM. Following a 5 min incubation, the test samples were placed into a 3 mm quartz cuvette, irradiated for 40 msec with an argon ion laser beam having a wavelength of 488 nm and monitored for fluorescent emission. The intensity of fluorescence was plotted as a function of wavelength for each sample analyzed.

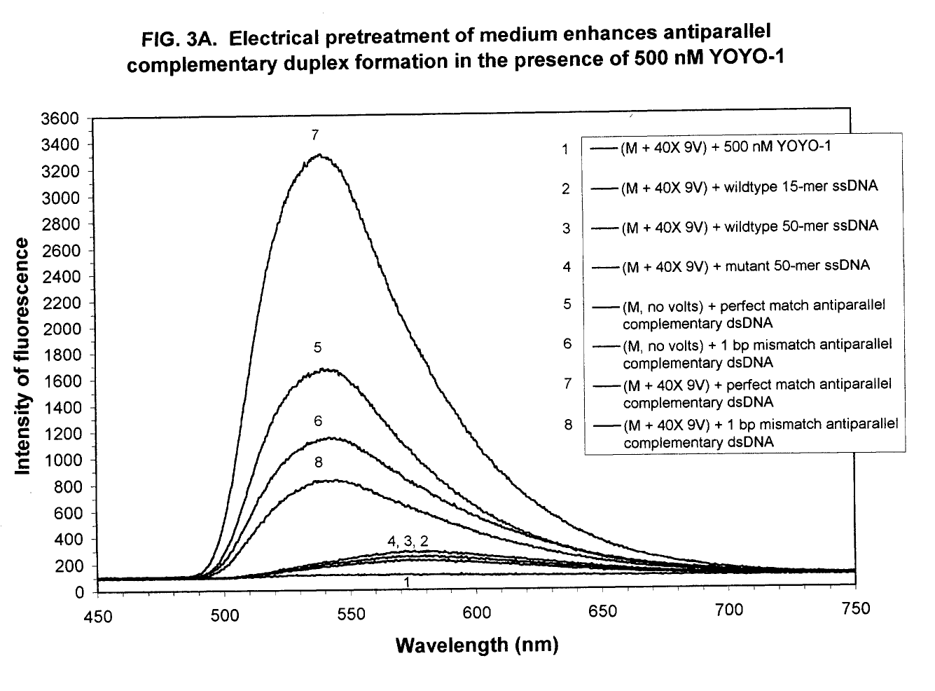

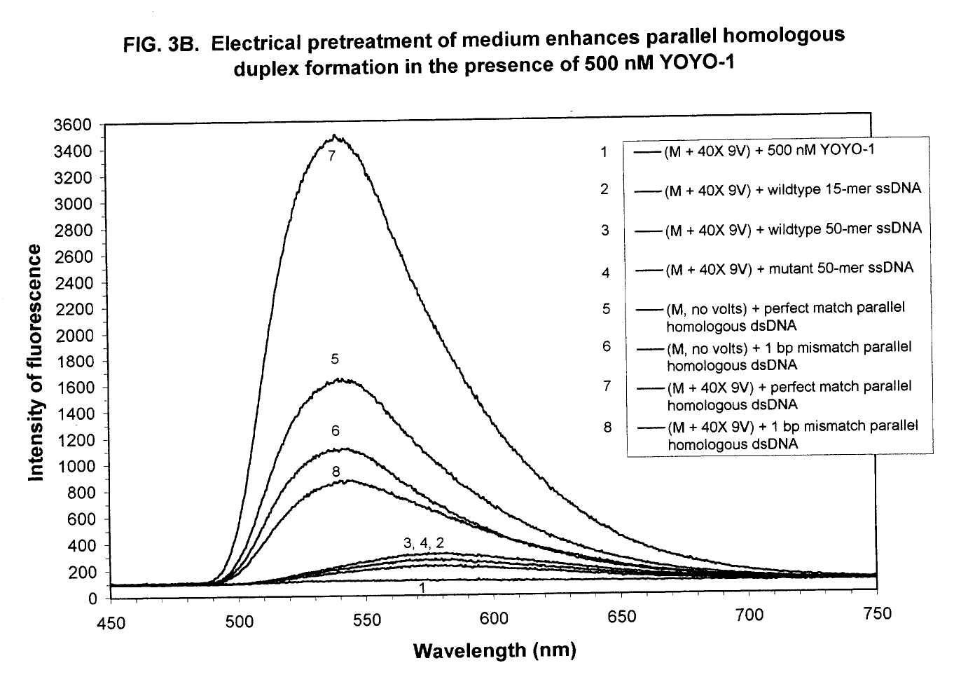

When the sense strand of the wild-type ssDNA probe (SEQ ID NO:3) was reacted with the 50-mer wild-type antisense strand of SEQ ID NO:4 or with the 50-mer mutant antisense strand of SEQ ID NO:5 in the presence of YOYO-1, antiparallel complementary ssDNA:ssDNA duplexes were formed (FIG. 3A). The fluorescent intensity emitted by the 1 bp mismatched antiparallel complementary duplex (sense strand of SEQ ID NO:3+antisense strand of SEQ ID NO:5) was 33.8% lower than that obtained by the perfectly matched antiparallel complementary duplex (sense strand of SEQ ID NO:3+antisense strand of SEQ ID NO:4).

The electrical pretreatment by the application of forty 500 msec pulses of 9V of DC current to the test medium dramatically increased perfectly matched antiparallel complementary duplex (sense strand of SEQ ID NO:3+antisense strand of SEQ ID NO:4) formation, resulting in a 104.0% increase in fluorescent emission compared to that observed with the same sample in non-treated test medium (FIG. 3A). In sharp contrast, electrical pretreatment with forty 9V pulses to the test medium suppressed 1 bp mismatched antiparallel complementary duplex (sense strand of SEQ ID NO:3+antisense strand of SEQ ID NO:5) formation, as indicated by a 31.1% decrease in fluorescence compared to that observed with the same mismatched sample in non-treated test medium (FIG. 3A). The fluorescent intensity emitted by the 1 bp mismatched antiparallel complementary duplex in pretreated medium was 77.5% lower than that obtained by the perfectly matched antiparallel complementary duplex. The electrical pretreatment of the test medium in the manner described preferentially promoted perfectly matched antiparallel complementary duplex formation in the presence of YOYO-1 and improved the specificity of binding by suppressing mismatch binding under like conditions.

Control samples comprising each 50-mer ssDNA target or 15-mer ssDNA probe plus 500 nM YOYO-1 exhibited levels of fluorescence that were just slightly greater than that produced by YOYO-1 alone both in the electrically pretreated samples (FIG. 3A) and in the untreated samples (data not shown).

When the sense strand of the wild-type ssDNA probe (SEQ ID NO:3) was reacted with the 50-mer wild-type sense strand of SEQ ID NO:4 in the presence of YOYO-1, the efficiency of parallel homologous ssDNA:ssDNA duplex formation was only 2.7% lower than the efficiency of antiparallel complementary ssDNA:ssDNA duplex formation (compare FIG. 3A and FIG. 3B). The 1 bp mismatched parallel homologous duplex formed when the sense strand of SEQ ID NO:3 was reacted with the 50-mer mutant sense strand of SEQ ID NO:5 in the presence of YOYO-1, produced a fluorescent emission intensity that was 34.5% lower than that emitted by the perfectly matched parallel homologous duplex (FIG. 3B). In the parallel homologous complexes, the 1 bp mismatch was a non-homologous base pair, which can be a Watson-Crick binding pair such as G:C or A:T.

The pretreatment of forty 500 msec 9V pulses to the test medium significantly enhanced perfectly matched parallel homologous duplex (sense strand of SEQ ID NO:3+sense strand of SEQ ID NO:4) formation, resulting in a 121.6% increase in fluorescent emission compared to that observed with the same sample in non-treated test medium (FIG. 3B). This voltage application reduced 1 bp mismatched parallel homologous duplex (sense strand of SEQ ID NO: 3+sense strand of SEQ ID NO: 5) formation, as indicated by a 24.5% decrease in fluorescence compared to that observed with the untreated sample (FIG. 3B). The 77.5% decrease in fluorescence emitted by the 1 bp mismatched parallel homologous duplex compared to that emitted by the perfectly matched parallel homologous duplex (FIG. 3B) demonstrated the increased rate and specificity of parallel homologous duplex binding following electrical pretreatment of the medium.

Control samples comprising each 50-mer ssDNA target or 15-mer ssDNA probe plus 500 nM YOYO-1 showed fluorescence levels that were marginally greater than that produced by YOYO-1 alone both in the electrically pretreated samples (FIG. 3B) and in the untreated samples (data not shown).

A set of forty 500 msec pulses of 9V was found to be an electrical pretreatment protocol which preferentially promoted subsequent perfectly matched antiparallel complementary duplex formation and perfectly matched parallel homologous duplex formation, while diminishing 1 bp mismatched duplex formation. Five, ten, twenty or thirty pulses of 9V also enhanced perfectly matched antiparallel complementary and parallel homologous duplexes, improving specificity between perfectly matched duplexes and 1 bp mismatched duplexes (data not shown). Increasing numbers of 9V pulses applied to pretreat the test medium led to greater enhancement of perfectly matched duplexes. Fifty pulses of 9V proved to be too many, resulting in less duplex formation and less discrimination between perfectly matched and 1 bp mismatched duplexes (data not shown).

### Example 4

Example 3 had demonstrated that electrical pretreatment of medium selectively promoted both perfectly matched antiparallel complementary duplex and perfectly matched parallel homologous duplex formation in the presence of YOYO-1. Example 4 investigated the effect of increasing the time between electrical pretreatment of the medium and subsequent addition of the DNA to the pretreated medium.

Forty 500 msec pulses of nine volts of DC current, separated by a 10 second interval between each pulse was applied to the test medium consisting of 0.7×TBE. The electrified medium was left to stand at room temperature for up to 45 min prior to the addition of two pmoles of the 15-mer ssDNA probe (sense strand of SEQ ID NO:3), two pmoles of the 50-mer ssDNA target and YOYO-1. The final volume of the untreated or electrically pretreated test media was 80 μl. The final buffer concentration was 0.5×TBE, and the final YOYO-1 concentration was 500 nM. Following a 5 min incubation of the reagents, the test samples were placed into a 3 mm quartz cuvette, irradiated for 60 msec with an argon ion laser beam having a wavelength of 488 nm and monitored for fluorescent emission. The intensity of fluorescence was plotted as a function of wavelength for each sample analyzed.

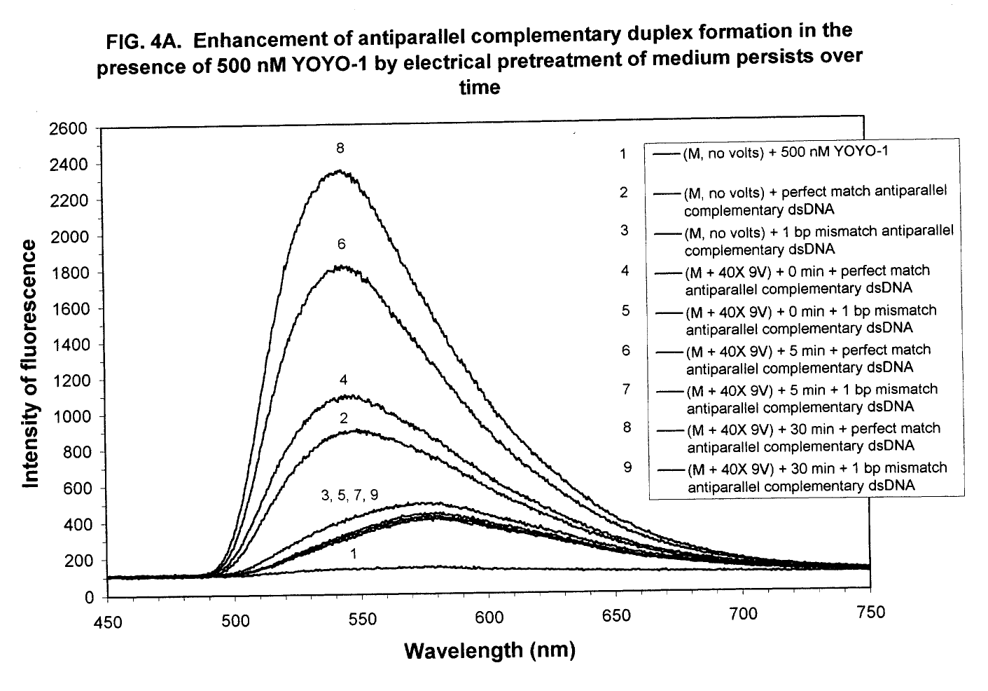

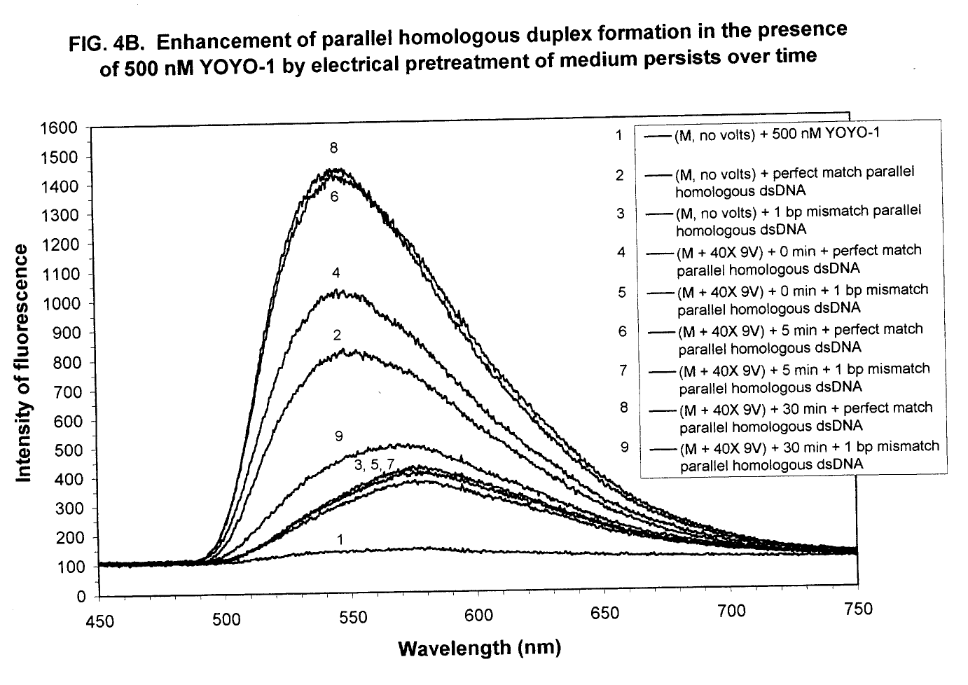

When the sense strand of the wild-type ssDNA probe (SEQ ID NO:3) was reacted with the 50-mer wild-type antisense strand of SEQ ID NO:4 or with the 50-mer mutant antisense strand of SEQ ID NO:5 in the presence of YOYO-1 in non-electrified medium, the fluorescent intensity emitted by the 1 bp mismatched antiparallel complementary duplex (sense strand of SEQ ID NO:3+antisense strand of SEQ ID NO:5) was 61.1% lower than that obtained by the perfectly matched antiparallel complementary duplex (sense strand of SEQ ID NO:3+antisense strand of SEQ ID NO:4) (FIG. 4A).

The pretreatment by forty 500 msec 9V pulses followed by immediate addition of both ssDNAs to the medium significantly enhanced perfectly matched antiparallel complementary duplex (sense strand of SEQ ID NO:3+antisense strand of SEQ ID NO:4) formation, resulting in a 24.2% increase in fluorescent emission compared to that observed with the same sample in untreated test medium (FIG. 4A). Letting the pretreated test medium stand for 5 minutes or 30 minutes, prior to addition of the sense strand of SEQ ID NO:3 and the antisense strand of SEQ ID NO:4, resulted in a 119.3% and 190.2% increase in fluorescent emission, respectively, compared to that observed with the same sample in non-electrified test medium, indicative of enhanced perfectly matched antiparallel complementary duplex formation in pretreated medium which had been allowed to stand before addition of the DNA (FIG. 4A). By comparison very little change in the level of fluorescence emitted and duplex formation were observed between the 1 bp mismatched antiparallel complementary duplexes (sense strand of SEQ ID NO:3+antisense strand of SEQ ID NO:5) present in the electrically pretreated media that were incubated for 0 min, 5 min or 30 min prior to addition of DNA (FIG. 4A). This translated to a 72.6%, a 83.7% and a 84.1% decrease in fluorescence observed between perfectly matched and 1 bp mismatched antiparallel complementary duplexes that were present in the electrically pretreated media that were incubated for 0 min, 5 min or 30 min, respectively, prior to addition of DNA (FIG. 4A). Increasing the time between electrical pretreatment of the medium and the time of addition of the complementary ssDNAs enhanced the specificity between perfectly matched and 1 bp mismatched antiparallel complementary duplex formation.

Similar levels of enhancement of perfectly matched antiparallel complementary duplex formation were observed after pretreated medium was allowed to stand for 40 min as for a 30 min incubation between electrically pretreated media and addition of DNA (data not shown). However, if 45 minutes elapsed between electrical pretreatment of the medium and the addition of reagents, perfectly matched antiparallel complementary duplex formation was reduced to a level below that achieved in untreated test medium (data not shown). These data demonstrate that the effect of electrical pretreatment of medium is progressive and only achieves its maximum or saturation level after a period of time, such as 40 minutes at room temperature.

An identical experiment was performed to evaluate the effect of increasing the elapsed time between electrical pretreatment of the medium and addition of reagents, on the enhancement of parallel homologous duplex formation. The 1 bp mismatched parallel homologous duplex formed when the sense strand of SEQ ID NO:3 was reacted with the 50-mer mutant sense strand of SEQ ID NO:5 in the presence of YOYO-1, produced a fluorescent emission intensity that was 59.1% lower than that emitted by the perfectly matched parallel homologous duplex (sense strand of SEQ ID NO:3+sense strand of SEQ ID NO:4) in an untreated test medium (FIG. 4B).

The application of forty 9V pulses of electrical pretreatment to the test medium followed by the immediate addition of both ssDNAs enhanced perfectly matched parallel homologous duplex (sense strand of SEQ ID NO:3+sense strand of SEQ ID NO:4) formation, resulting in a 27.7% increase in fluorescent emission compared to that observed with the same sample in untreated test medium (FIG. 4B). Allowing the electrically pretreated medium to stand for 5 minutes, 30 minutes or 40 minutes, prior to addition of the sense strand of SEQ ID NO:3 and the antisense strand of SEQ ID NO:4, resulted in a 86.0%, a 90.3% and a 109.3% increase in fluorescent emission, respectively, compared to that observed with the same sample in untreated test medium (FIG. 4B and data not shown). These results demonstrated enhanced perfectly matched parallel homologous duplex formation with increased elapse time between electrical pretreatment of the medium and addition of the reagents. The level of fluorescence slightly decreased in the 1 bp mismatched antiparallel complementary duplexes (sense strand of SEQ ID NO:3+antisense strand of SEQ ID NO:5) present in the electrically pretreated medium that were allowed to stand for 0 min or 5 min prior to addition of DNA (FIG. 4B). When 30 min or 40 min elapsed before addition of reagents however, a 26.4% or 17.3% increase, respectively, in fluorescence was observed for the 1 bp mismatched parallel homologous duplexes. This corresponded to a 71.5%, a 82.6%, a 72.9% and a 77.1% decrease in fluorescence observed between perfectly matched and 1 bp mismatched parallel homologous duplexes that were formed in the electrically pretreated media after an elapse time of 0 min, 5 min, 30 min or 40 min, respectively, prior to addition of DNA (FIG. 4B and data not shown). Therefore the modification of the medium caused by electrical pretreatment increased for at least 40 min, resulting in an enhancement of perfectly matched parallel homologous duplex subsequently formed in the medium. However, after 45 min of delay in adding reagents to the pretreated medium, perfectly matched parallel homologous duplex formation was reduced to a level below that achieved in non-electrified test medium (data not shown).

### Example 5

Example 5 identifies a specific electrical treatment protocol that reduces the specificity of antiparallel complementary duplex binding in the presence of YOYO-1, while increasing the specificity of parallel homologous duplex binding. Perfectly matched parallel homologous duplex binding is enhanced while 1 bp mismatched parallel homologous duplex binding is suppressed.

Ten 500 msec pulses of nine volts of DC current, separated by a 30 second interval between each pulse was applied to the test medium consisting of 0.7×TBE and two pmoles of the 15-mer ssDNA probe (sense strand of SEQ ID NO:3). The electrified medium and ssDNA probe were left to stand at room temperature for 5 min prior to the addition of two pmoles of the 50-mer ssDNA target and YOYO-1. The final volume of the untreated or electrically pretreated test media was 80 μl. The final buffer concentration was 0.5×TBE, and the final YOYO-1 concentration was 500 nM. Following a 5 min incubation of all of the reagents, the test samples were placed into a 3 mm quartz cuvette, irradiated for 200 msec with an argon ion laser beam having a wavelength of 488 nm and monitored for fluorescent emission. The intensity of fluorescence was plotted as a function of wavelength for each sample analyzed.

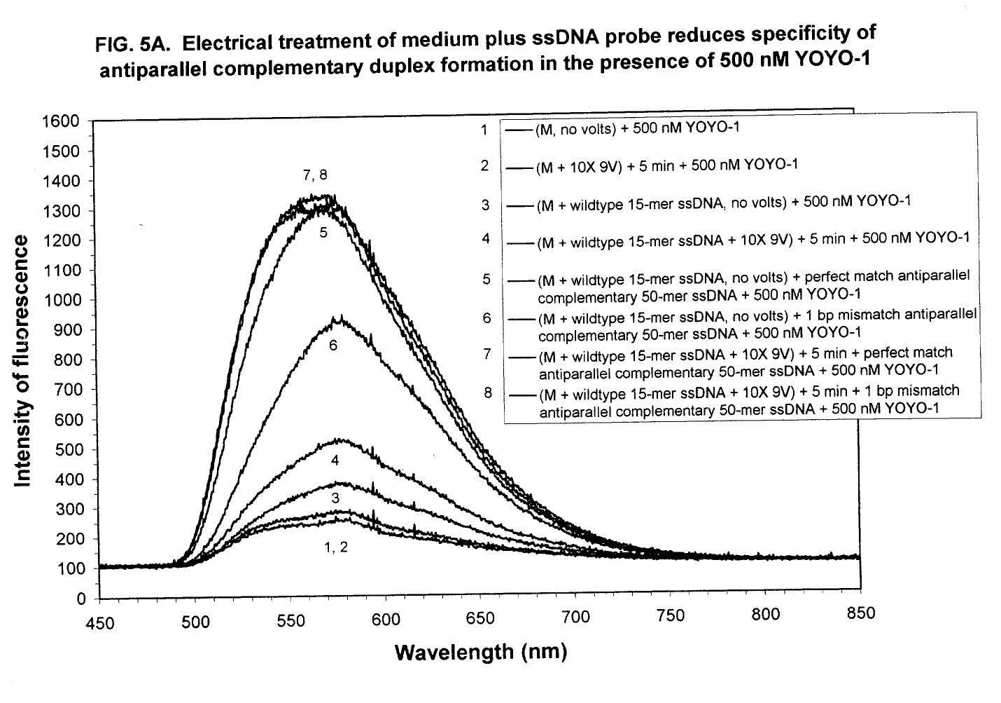

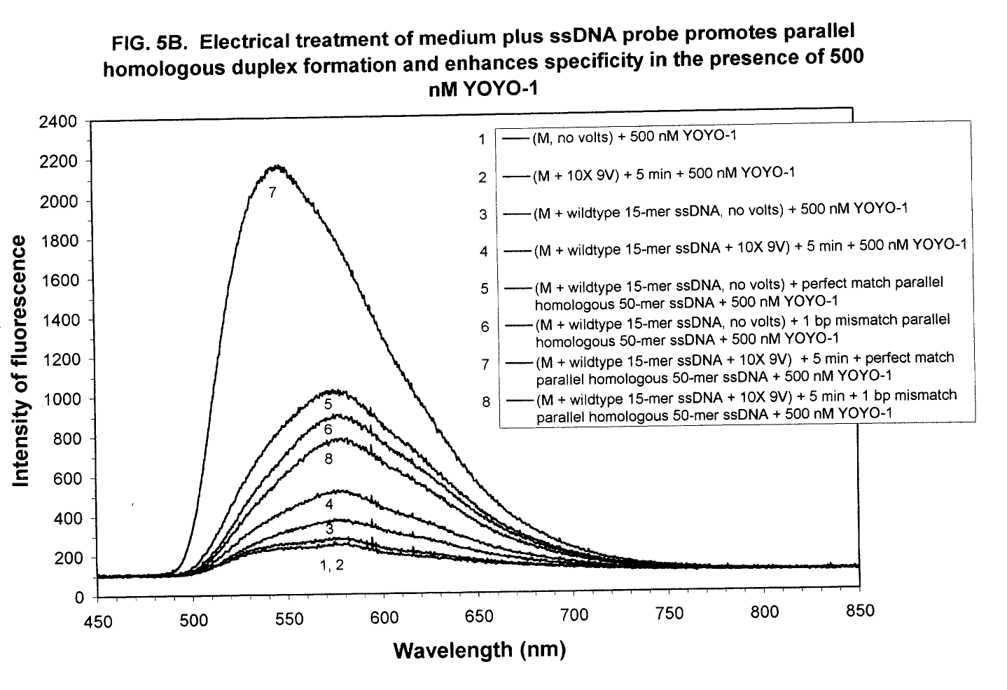

When the sense strand of the wild-type ssDNA probe (SEQ ID NO:3) was reacted with the 50-mer wild-type antisense strand of SEQ ID NO:4 or with the 50-mer mutant antisense strand of SEQ ID NO:5 in the presence of YOYO-1 in untreated medium, the fluorescent intensity emitted by the 1 bp mismatched antiparallel complementary duplex (sense strand of SEQ ID NO:3+antisense strand of SEQ ID NO:5) was 37.9% lower than that obtained by the perfectly matched antiparallel complementary duplex (sense strand of SEQ ID NO:3+antisense strand of SEQ ID NO:4) (FIG. 5A).

The application of ten 9V pulses of electrical treatment to the test medium containing the wild-type ssDNA probe followed by a 5 min incubation prior to the addition of the 50-mer wild-type antisense strand of SEQ ID NO:4 only marginally augmented perfectly matched antiparallel complementary duplex (sense strand of SEQ ID NO:3+antisense strand of SEQ ID NO:4) formation, resulting in a 5.5% increase in fluorescent emission compared to that observed with the same sample in untreated test medium (FIG. 5A). In contrast, this same electrical treatment protocol significantly enhanced 1 bp mismatched antiparallel complementary duplex (sense strand of SEQ ID NO:3+antisense strand of SEQ ID NO:5) formation, as indicated by a 103.9% increase in fluorescence compared to that observed with the same mismatched sample in non-treated test medium (FIG. 5A). The fluorescent intensity emitted by the 1 bp mismatched antiparallel complementary duplex in the electrified medium containing the sense strand of SEQ ID NO:3 was only 3.4% lower than that obtained by the perfectly matched antiparallel complementary duplex. Specificity of antiparallel complementary duplex binding was thus lost.

The 1 bp mismatched parallel homologous duplex formed when the sense strand of SEQ ID NO:3 was reacted with the 50-mer mutant sense strand of SEQ ID NO:5 in the presence of YOYO-1 in the untreated medium, produced a fluorescent emission intensity that was 15.8% lower than that emitted by the perfectly matched parallel homologous duplex (sense strand of SEQ ID NO:3+sense strand of SEQ ID NO:4) formed in the untreated medium (FIG. 5B).

Perfectly matched parallel homologous duplex formation was significantly enhanced by the application of ten 9V pulses of electrical treatment to the test medium containing the sense strand of SEQ ID NO:3 followed by a 5 min incubation prior to the addition of the 50-mer wild-type sense strand of SEQ ID NO:4, as indicated by the 157.0% increase in fluorescent emission compared to that observed with the same sample in untreated medium (FIG. 5B). This voltage treatment moreover reduced 1 bp mismatched parallel homologous duplex (sense strand of SEQ ID NO:3+sense strand of SEQ ID NO:5) formation, resulting in a 15.3% decrease in fluorescence compared to that observed when the reaction took place in an untreated sample (FIG. 5B). The resultant 72.2% decrease in fluorescence emitted by the 1 bp mismatched parallel homologous duplex compared to that emitted by the perfectly matched parallel homologous duplex demonstrated the vastly improved specificity of parallel homologous duplex binding achieved as a result of this electrical treatment protocol.

The application of ten 9V pulses of electrical treatment to the test medium containing the ssDNA probe followed by a 5 min incubation prior to the addition of the complementary or homologous ssDNA target, reduced the specificity of antiparallel complementary duplex binding in the presence of YOYO-1, while increasing the specificity of parallel homologous duplex binding by selectively promoting perfectly matched parallel homologous duplex binding and suppressing 1 bp mismatched parallel homologous duplex binding.

### Example 6

Example 6 demonstrates a specific electrical treatment protocol that reduces both the level and the specificity of parallel homologous duplex binding, while increasing the specificity of antiparallel complementary duplex binding by selectively promoting perfectly matched antiparallel complementary duplex binding and suppressing 1 bp mismatched antiparallel complementary duplex binding.

Two pmoles of the 15-mer ssDNA probe (sense strand of SEQ ID NO:3) were reacted at room temperature with two pmoles of the 50-mer ssDNA target and 500 nM YOYO-1 in 0.5×TBE for 5 minutes. Duplicate samples of 80μl were placed into a 3 mm quartz cuvette, irradiated for 60 msec with an argon ion laser beam having a wavelength of 488 nm and monitored for fluorescent emission. Half of the samples remained untreated and were irradiated for 60 msec for a second time 5 minutes after the first irradiation. The other half of the duplicate samples were electrified with a single 500 msec five volt DC pulse and irradiated for 60 msec for a second time 5 minutes after the first irradiation. The intensity of fluorescence was plotted as a function of wavelength for each sample analyzed.

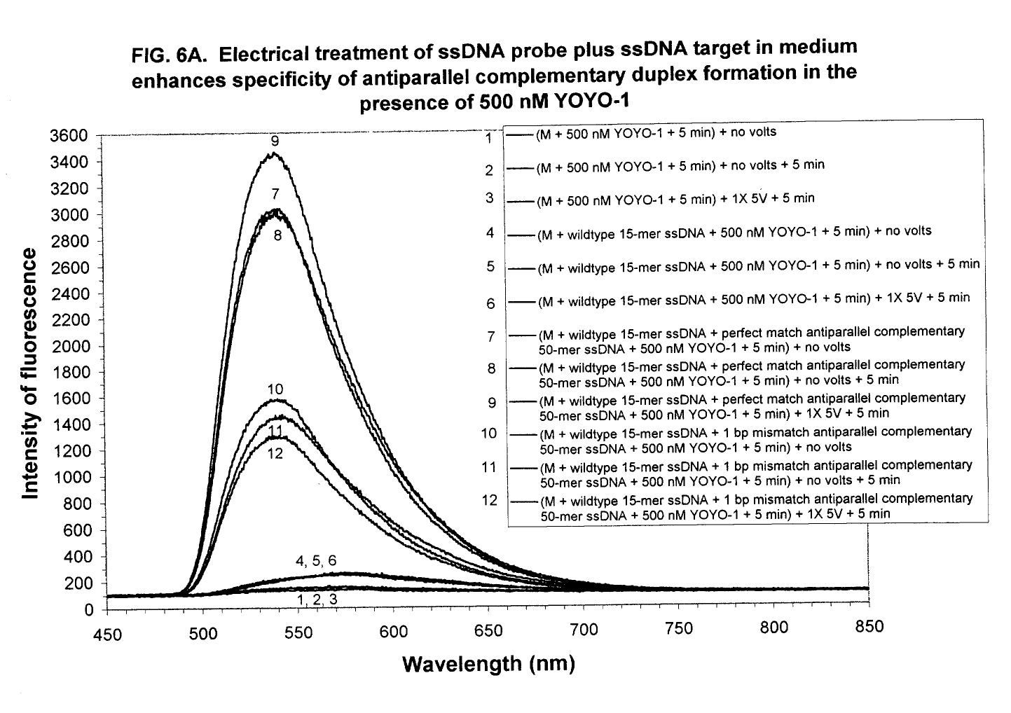

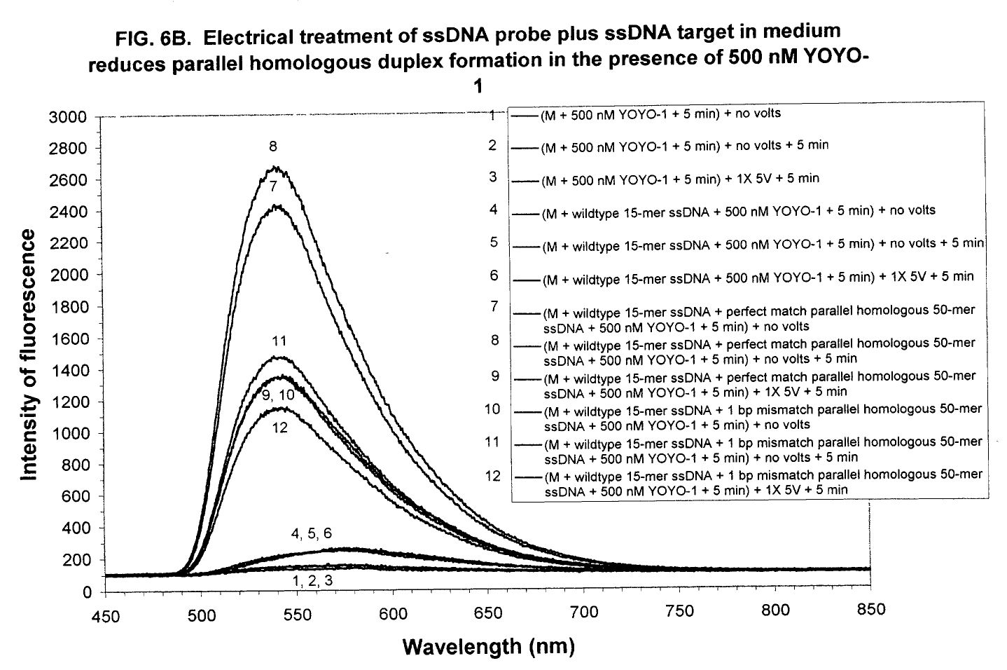

Nearly identical levels of fluorescence were observed when the sense strand of the wild-type ssDNA probe (SEQ ID NO:3) was allowed to bind for 5 minutes or 10 minutes with the 50-mer wild-type antisense strand of SEQ ID NO:4, indicating that the level of perfectly matched antiparallel complementary duplex formation that had occurred within the first 5 minutes in the untreated medium was essentially stable for the next 5 minutes (FIG. 6A). Application of a single 5V pulse between the first irradiation at 5 minutes and the second irradiation at 10 minutes resulted in enhanced perfectly matched antiparallel complementary duplex formation and an increase in fluorescence emission of 14.7% (FIG. 6A).

The fluorescent intensity emitted by the 1 bp mismatched antiparallel complementary duplex (sense strand of SEQ ID NO:3+antisense strand of SEQ ID NO:5) in the untreated medium decreased by 7.8% between the 5 min and the 10 min measurements, indicating a slight decrease in stability of duplex binding (FIG. 6A). These results translated to a 50.5% and a 54.2% decrease in fluorescence observed between perfectly matched and 1 bp mismatched antiparallel complementary duplexes that were present in untreated medium after a 5 min and a 10 min incubation, respectively. Application of a single 5V pulse between the first irradiation at 5 minutes and the second irradiation at 10 minutes resulted in a 20.1% decrease in fluorescence indicative of a significantly reduced 1 bp mismatched antiparallel complementary duplex binding (FIG. 6A). As a consequence, the level of fluorescence between the perfectly matched antiparallel complementary duplex and the 1 bp mismatched antiparallel complementary duplex in the electrically treated medium had decreased by 65.5% between the 5 min and the 10 min measurements. Therefore, this electrical treatment protocol had enhanced the specificity of antiparallel complementary duplex binding.

The level of fluorescence in the control samples containing only the sense strand of SEQ ID NO:3, the test medium and YOYO-1, did not change with time or as a result of electrical treatment in the time between the two laser irradiations (FIG. 6A and FIG. 6B).

When the sense strand of the wild-type ssDNA probe (SEQ ID NO:3) was allowed to incubate for 5 minutes or 10 minutes with the 50-mer wild-type sense strand of SEQ ID NO:4, a slight increase (11.5%) in the level of fluorescence was observed, indicating that the level of perfectly matched parallel homologous duplex formation in the untreated medium had slightly increased between the 5 min and 10 min measurements (FIG. 6B). In sharp contrast, application of a single 5V pulse between the first irradiation at 5 minutes and the second irradiation at 10 minutes resulted in a dramatic suppression of perfectly matched parallel homologous duplex binding, as evidenced by the 47.3% decrease in fluorescence emission (FIG. 6B).

One bp mismatched parallel homologous duplex (sense strand of SEQ ID NO:3+sense strand of SEQ ID NO:5) binding increased slightly during the 5 min incubation in the untreated medium between the first irradiation and the second irradiation, resulting in a 12.0% increase in fluorescence (FIG. 6B). This corresponded to a 47.5% and a 47.3% decrease in fluorescence observed between perfectly matched and 1 bp mismatched parallel homologous duplexes that had formed in untreated medium after a 5 min and a 10 min incubation, respectively.

Application of a single 5V pulse between the first irradiation at 5 minutes and the second irradiation at 10 minutes resulted in a 16.7% decrease in fluorescence indicative of diminished 1 bp mismatched parallel homologous duplex binding (FIG. 6B). The level of fluorescence between the perfectly matched parallel homologous duplex and the 1 bp mismatched parallel homologous duplex in the electrically treated medium had decreased only 17.0% between the 5 min and the 10 min measurements. This was a dramatic change in specificity of parallel homologous duplex formation.

Perfectly matched and 1 bp mismatched parallel homologous duplexes, that had formed within 5 minutes of incubation in the presence of YOYO-1, were dramatically destabilized by the application of a single 500 msec 5V pulse. This voltage treatment also significantly reduced the specificity of parallel homologous duplex binding. The same voltage application administered to preformed antiparallel complementary duplexes however increased the specificity of antiparallel complementary duplex binding by selectively promoting perfectly matched antiparallel complementary duplex binding while suppressing 1 bp mismatched antiparallel complementary duplex binding.

The application of five 500 msec pulses of 3V (separated by 30 seconds between each pulse) to preformed parallel homologous duplexes, also resulted in significant inhibition of the level and specificity of parallel homologous duplex binding (data not shown). The same voltage application administered to preformed antiparallel complementary duplexes, also significantly enhanced perfectly matched antiparallel complementary duplex binding and increased the specificity of antiparallel complementary duplex binding (data not shown).

### Example 7

The previous Examples demonstrated the effect of various electrical treatment protocols on antiparallel complementary duplex formation and parallel homologous duplex formation. Example 7 investigates the effect of electrical pretreatment of medium on Watson-Crick triplex DNA binding between ssDNA probes and non-denatured dsDNA targets of mixed base sequence.

Forty 500 msec pulses of nine volts of DC current, separated by 10 second intervals were applied to 56 μl of test medium consisting of 0.7×TBE. Immediately after the final pulse of DC current, two pmoles of the 15-mer ssDNA probe (antisense strand of SEQ ID NO:3), two pmoles of the 50-mer dsDNA target and YOYO-1 were added to the untreated and electrically pretreated test media to give a final volume of 80 μl. The final buffer concentration was 0.5×TBE, and the final YOYO-1 concentration was 500 nM. Following a 5 min incubation, the test samples were placed into a 3 mm quartz cuvette, irradiated for 400 msec with an argon ion laser beam having a wavelength of 488 nm and monitored for fluorescent emission. The intensity of fluorescence was plotted as a function of wavelength for each sample analyzed.

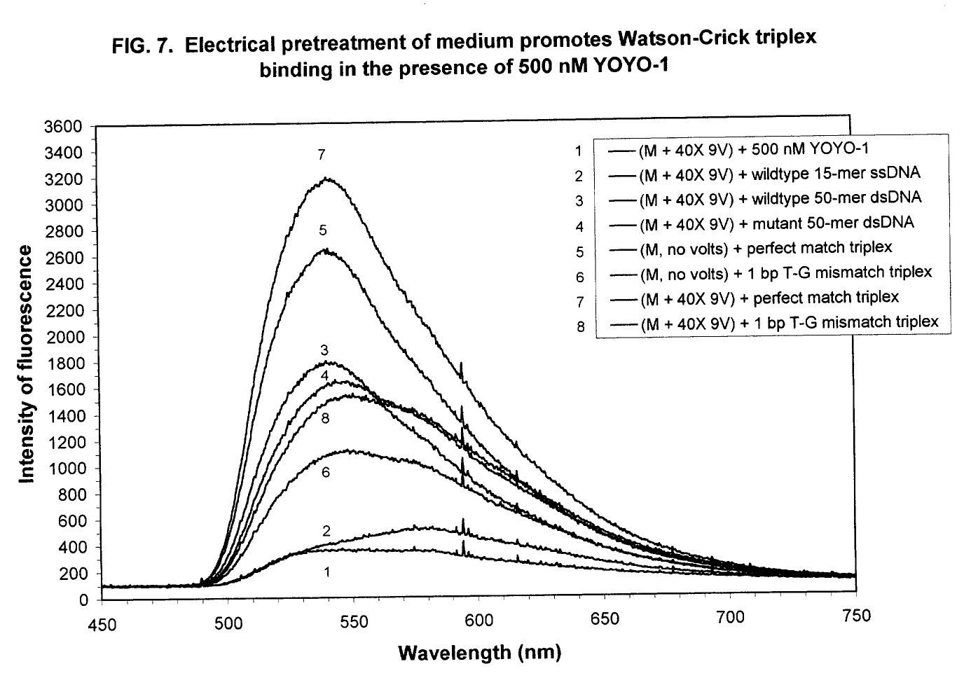

When the wild-type 15-mer ssDNA probe (antisense strand of SEQ ID NO:3) was reacted with the 50-mer wild-type dsDNA target (SEQ ID NO:4) and 50-mer mutant dsDNA target (SEQ ID NO:5), dsDNA:ssDNA complexes were formed in the untreated medium at room temperature under non-denaturing conditions (FIG. 7). While perfectly matched DNA complexes (antisense strand of SEQ ID NO:3+SEQ ID NO:4) emitted the highest fluorescent intensity, incompletely complementary complexes with a 1 bp T-G mismatch (antisense strand of SEQ ID NO:3+SEQ ID NO:5) produced a fluorescent intensity that was 64.6% lower than that observed with the perfectly matched complexes (FIG. 7). When the maximum fluorescent intensity values were normalized to account for triplex formation only, by subtracting the values of fluorescent intensities obtained for each dsDNA target, the 1 bp T-C mismatched dsDNA:ssDNA triplexes produced a fluorescent intensity that was more than 100% lower than that achieved by the perfectly matched triplexes. The level of fluorescence generated by the ssDNA probe plus 500 nM YOYO-1 was slightly above that obtained by YOYO-1 alone in the untreated medium (data not shown).

Electrical pretreatment with forty 9V pulses to the test medium enhanced perfectly matched Watson-Crick triplex (antisense strand of SEQ ID NO:3+SEQ ID NO:4) formation, resulting in a 19.7% increase in fluorescent emission compared to that observed with the same sample in untreated test medium (FIG. 7). One bp T-G mismatched triplex (antisense strand of SEQ ID NO:3+SEQ ID NO:4) formation was also increased by the application of forty 9V pulses to the test medium, such that the fluorescent intensity emitted by the 1 bp mismatched complexes in the pretreated medium was only 58.8% lower than that obtained by the perfectly matched complexes (FIG. 7). When the maximum fluorescent intensity values were normalized to account for triplex formation only, the 1 bp T-G mismatched dsDNA:ssDNA triplexes produced a fluorescent intensity that was more than 100% lower than that achieved by the perfectly matched triplexes in the pretreated medium. Control samples comprising the ssDNA probe plus 500 nM YOYO-1 exhibited levels of fluorescence which were just above that obtained by YOYO-1 alone in the pretreated medium (FIG. 7).

The application of sixty 9V pulses to the test medium also promoted both perfectly matched and 1 bp T-G mismatched triplex formation, such that the fluorescent intensity emitted by the 1 bp mismatched complexes in the pretreated medium was only 56.0% lower than that obtained by the perfectly matched complexes (data not shown).

Electrical pretreatment of the test medium enhanced Watson-Crick triplex binding between ssDNA probes and non-denatured wild-type or mutant dsDNA targets of mixed base sequence, without significantly altering the specificity of triplex binding. Nearly the same level of discrimination between perfectly matched triplexes and 1 bp mismatched triplexes was observed when reacted in the untreated medium or in the electrically pretreated medium.

### Example 8

Example 8 examines the effect of electrical pretreatment of medium on both Watson-Crick quadruplex binding and parallel homologous quadruplex binding between non-denatured dsDNA probes and non-denatured dsDNA targets of mixed base sequence.

Complementary sense and antisense 15-mer and 50-mer ssDNA sequences were synthesized, purified by HPLC and annealed as above to generate 15-mer dsDNA probes and 50-mer dsDNA targets. DsDNA probes were diluted in ddH₂O to a concentration of 1 pmole/μl. DsDNA targets were diluted in ddH₂O to a concentration of 0.1 pmole/μl.

Sequence for the sense strand of the wild-type 15-mer DNA probe (SEQ ID NO:3): 5′-CTG TCA TCT CTG GTG-3′

Sequence for the antisense strand of the wild-type 15-mer DNA probe (SEQ ID NO:3): 5′-CAC CAG AGA TGA CAG-3′

SEQ ID NO:4 was a 50-mer dsDNA target sequence. A central region of SEQ ID NO:4 was identical in sequence to that of SEQ ID NO:3.

Sequence for the sense strand of the wild-type target DNA (SEQ ID NO:4): 5′-GAG CAC CAT GAC AGA CAC TGT CAT CTC TGG TGT GTC CTA CGA TGA CTC TG-3′

Sequence for the antisense strand of the wild-type target DNA (SEQ ID NO:4): 5′-CAG AGT CAT CGT AGG ACA CAC CAG AGA TGA CAG TGT CTG TCA TGG TGC TC-3′

SEQ ID NO:5 was a 50-mer mutant dsDNA target sequence identical to SEQ ID NO:4, except for a one base pair mutation (underlined), at which the sequence CTC was changed to CTT.

Sequence for the sense strand of mutant SEQ ID NO:5: 5′-GAG CAC CAT GAC AGA CAC TGT CAT CTT TGG TGT GTC CTA CGA TGA CTC TG-3′

Sequence for the antisense strand of mutant SEQ ID NO:5: 5′-CAG AGT CAT CGT AGG ACA CAC CAA AGA TGA CAG TGT CTG TCA TGG TGC TC-3′

SEQ ID NO:6 was a 50-mer mutant dsDNA target sequence identical to SEQ ID NO:4, except for a one base pair mutation (underlined), at which the sequence CAT was changed to CTT.

Sequence for the sense strand of mutant SEQ ID NO:6: 5′-GAG CAC CAT GAC AGA CAC TGT CTT CTC TGG TGT GTC CTA CGA TGA CTC TG-3′

Sequence for the antisense strand of mutant SEQ ID NO:6: 5′-CAG AGT CAT CGT AGG ACA CAC CAG AGA AGA CAG TGT CTG TCA TGG TGC TC-3′

SEQ ID NO:7 was a 50-mer mutant dsDNA target sequence identical to SEQ ID NO:4, except for a one base pair mutation (underlined), at which the sequence CTC was changed to CCC.

Sequence for the sense strand of mutant SEQ ID NO:7: 5′-GAG CAC CAT GAC AGA CAC TGT CAT CCC TGG TGT GTC CTA CGA TGA CTC TG-3′

Sequence for the antisense strand of mutant SEQ ID NO:7: 5′-CAG ,AGT CAT CGT AGG ACA CAC CAG GGA TGA CAG TGT CTG TCA TGG TGC TC-3′

SEQ ID NO:8 was a 50-mer mutant dsDNA target sequence identical to SEQ ID NO:4, except for a one base pair mutation (underlined), at which the sequence CAT was changed to CGT.

Sequence for the sense strand of mutant SEQ ID NO:8: 5′-GAG CAC CAT GAC AGA CAC TGT CGT CTC TGG TGT GTC CTA CGA TGA CTC TG-3′

Sequence for the antisense strand of mutant SEQ ID NO:8: 5′-CAG AGT CAT CGT AGG ACA CAC CAG AGA CGA CAG TGT CTG TCA TGG TGC TC-3′

Sequence for the sense strand of the wild-type 15-mer DNA probe (SEQ ID NO:9): 5′-GAC AGT AGA GAC CAC-3′

Sequence for the antisense strand of the wild-type 15-mer DNA probe (SEQ ID NO:9): 5′-GTG GTC TCT ACT GTC-3′

Forty 500 msec pulses of nine volts of DC current, separated by a 10 second interval between each pulse was applied to 68 μl of test medium consisting of 0.6×TBE. Immediately after the final pulse of DC current, 2 pmoles of the 15-mer dsDNA probe, 0.2 pmoles of the 50-mer dsDNA target and YOYO-1 were added to the untreated and electrically pretreated test media to give final volumes of 80 μl. The final buffer concentrations were 0.5×TBE, and the final YOYO-1 concentrations were 100 nM. Following a 5 min incubation at room temperature, the test samples were placed into a 3 mm quartz cuvette, irradiated for 200 msec with an argon ion laser beam having a wavelength of 488 nm and monitored for fluorescent emission. The intensity of fluorescence was plotted as a function of wavelength for each sample analyzed.

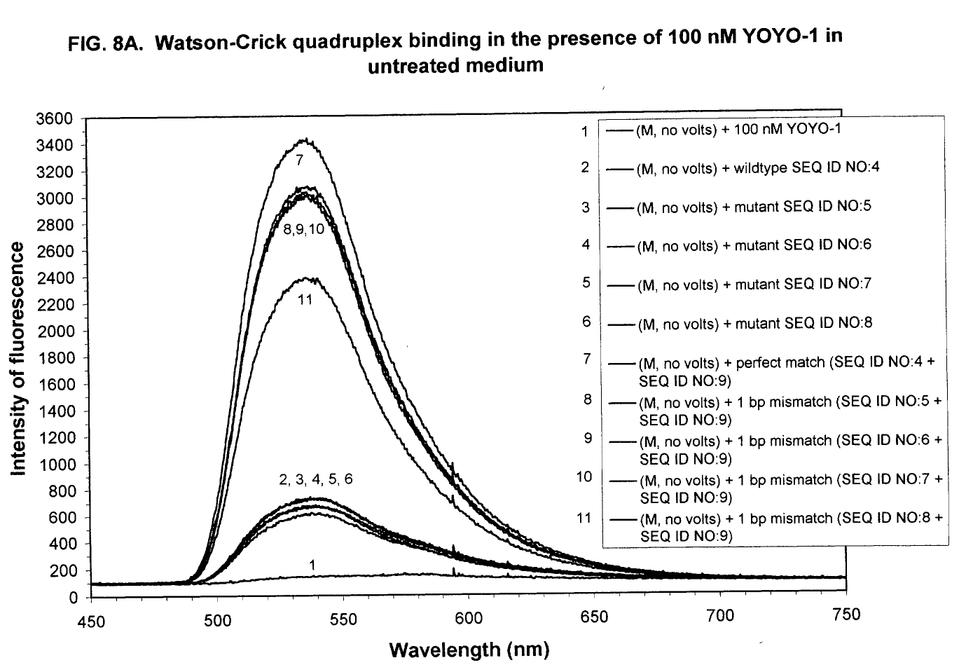

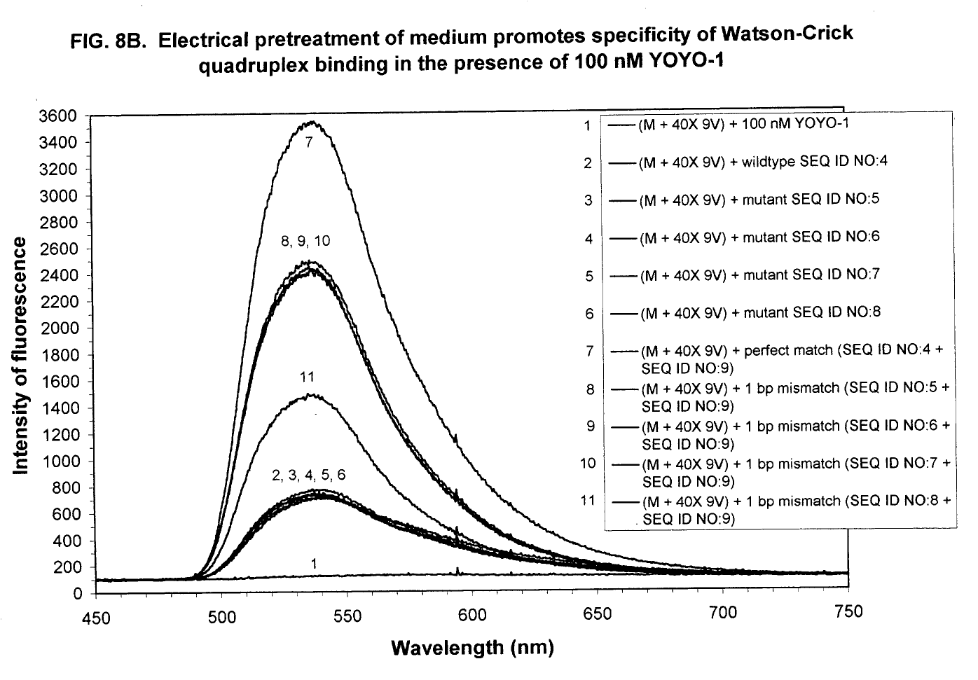

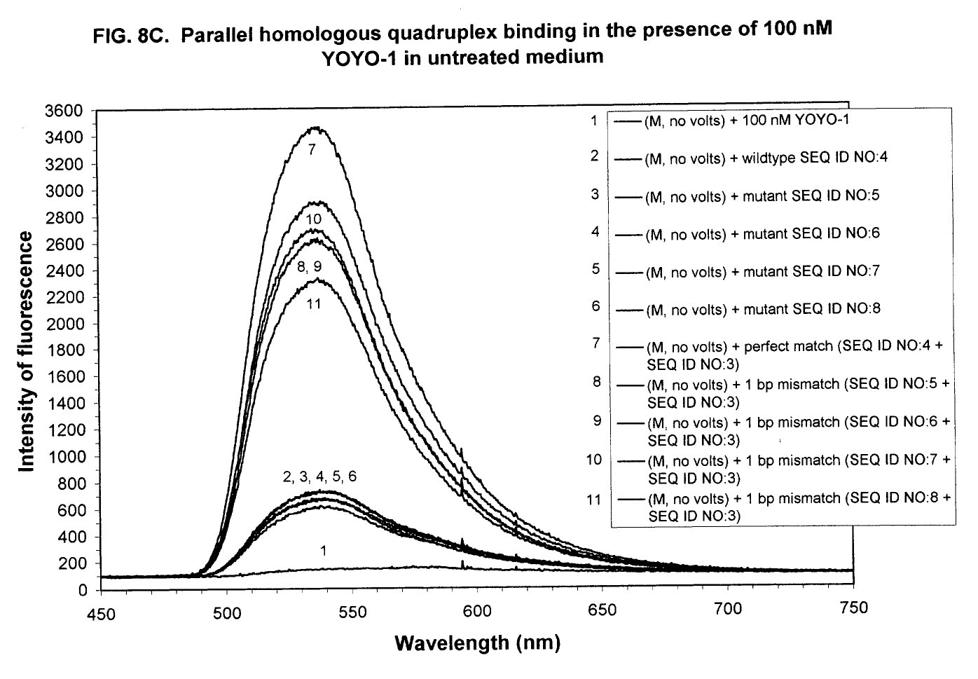

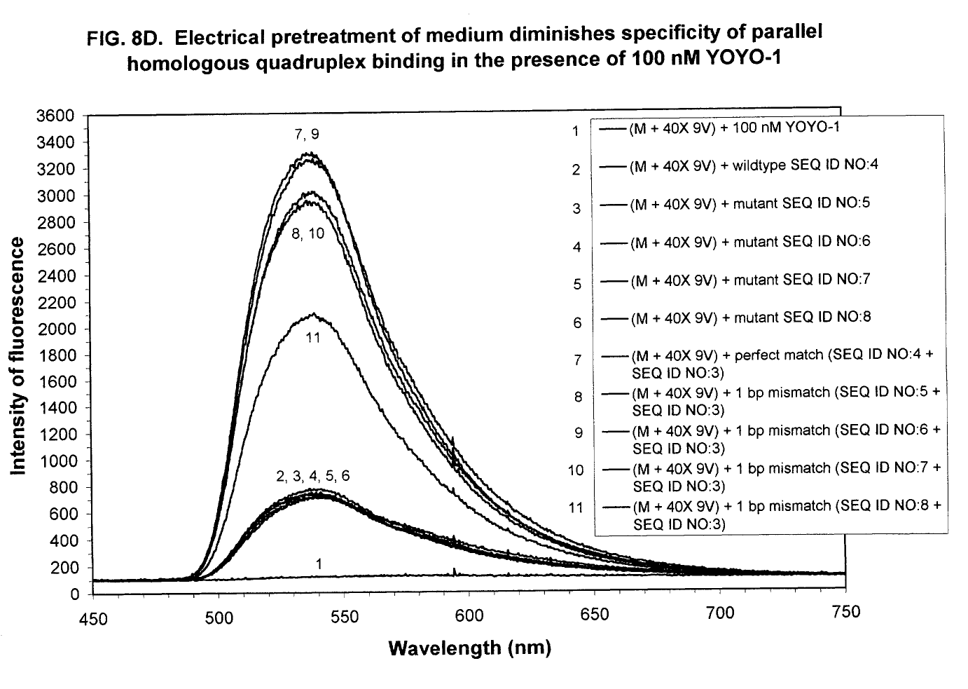

When the wild-type 15-mer dsDNA probe (SEQ ID NO:9) was reacted with the 50-mer wild-type dsDNA target (SEQ ID NO:4) and the 50-mer mutant dsDNA targets (SEQ ID NO:5 to SEQ ID NO:8), dsDNA:dsDNA complexes were formed in the untreated medium at room temperature under non-denaturing conditions (FIG. 8A). In these quadruplex complexes, Watson-Crick complementarity exists between bases of the strands of the probe and proximal bases of the strands of the target when the major groove of one duplex is placed in the minor groove of the other duplex. The sequences of bases in the duplex probe are not homologous but are inverted in relation to those in the duplex target. The duplexes, when nested major groove into minor groove, are parallel to one another, and referred to by us as nested complementary.

The highest fluorescent intensities were achieved when the wild-type 50-mer dsDNA target (SEQ ID NO:4) was reacted with the 15-mer dsDNA probe (SEQ ID NO:9), which was a perfect match on a nested complementary basis to the dsDNA target. The fluorescent intensity is indicative of DNA binding taking place, in this case quadruplex formation between the dsDNA target and the nested complementary dsDNA probe.

Mutant dsDNA targets which were mismatched with the duplex probe by a single pair of bases when matching was assessed based on nested complementarity, formed measurably fewer quadruplex complexes with the dsDNA probe, than did the fully complementary wild-type dsDNA target. As shown in FIG. 8A, the fluorescent intensities produced by the quadruplexes formed with the 1 bp mismatched dsDNA targets plus the dsDNA probe (SEQ ID NO:9), ranged from 10.8% to 31.9% less than that achieved by the perfectly matched quadruplexes. When the maximum fluorescent intensity values were normalized by subtracting the fluorescent intensity value obtained for the dsDNA probe (SEQ ID NO:9), the mismatched quadruplexes comprised of SEQ ID NO:5+SEQ ID NO:9, SEQ ID NO:6+SEQ ID NO:9, SEQ ID NO:7+SEQ ID NO:9 or SEQ ID NO:8+SEQ ID NO:9 emitted fluorescent intensities that were 56.9%, 50.7%, 60.5% and 100% lower, respectively, than that achieved by the perfectly matched quadruplexes (SEQ ID NO:4+SEQ ID NO:9) in the untreated medium (FIG. 8A). Control samples comprising each dsDNA target plus 100 nM YOYO-1 exhibited similar levels of fluorescence, which constituted no more than 17.6% of the fluorescent level achieved by the perfectly matched quadruplex (FIG. 8A).

Electrical pretreatment with forty 9V pulses to the test medium mildly enhanced perfectly matched Watson-Crick quadruplex (SEQ ID NO:4+SEQ ID NO:9) formation (FIG. 8B), resulting in a 4.2% increase in fluorescent emission compared to that observed with the same sample in untreated test medium (FIG. 8A). Mismatched quadruplex formation decreased due to the application of forty 9V pulses to the test medium, such that the fluorescent intensities emitted by the 1 bp mismatched complexes in the pretreated medium were 30.9% to 60.2% lower than that obtained by the perfectly matched complexes (FIG. 8B). When the maximum fluorescent intensity values were normalized by subtracting the fluorescent intensity value obtained for the dsDNA probe (SEQ ID NO:9), the mismatched quadruplexes comprised of SEQ ID NO:5+SEQ ID NO:9, SEQ ID NO:6+SEQ ID NO:9, SEQ ID NO:7+SEQ ID NO:9 or SEQ ID NO:8+SEQ ID NO:9 emitted fluorescent intensities that were all 100% lower than that achieved by the perfectly matched quadruplexes (SEQ ID NO:4+SEQ ID NO:9) in the pretreated medium (FIG. 8B). Control samples comprising each dsDNA target plus 100 nM YOYO-1 in pretreated medium exhibited similar levels of fluorescence that constituted no more than 18.8% of the fluorescent level achieved by the perfectly matched quadruplex (FIG. 8B).

Electrical pretreatment of the test medium significantly enhanced the specificity of Watson-Crick quadruplex binding, as a result of greatly diminished levels of mismatched quadruplex formation signal as compared to signal levels generated upon perfectly complementary quadruplex formation.

When the wild-type 15-mer dsDNA probe (SEQ ID NO:3) was reacted with the 50-mer wild-type dsDNA target (SEQ ID NO:4) and the 50-mer mutant dsDNA targets (SEQ ID NO:5 to SEQ ID NO:8), parallel homologous dsDNA:dsDNA complexes were formed in the untreated medium at room temperature under non-denaturing conditions (FIG. 8C). Perfectly matched parallel homologous quadruplexes formed as efficiently as perfectly matched nested complementary quadruplexes (compare FIG. 8C and FIG. 8A).

It was observed that parallel homologous quadruplexes, stabilized by YOYO-1 intercalation, formed more readily between a dsDNA target and a dsDNA probe when that probe contained perfectly homologous sequences, than when there was a single pair of bases which were not homologous, that is to say identical, to a pair of bases in the dsDNA target. As illustrated in FIG. 8C, the fluorescent intensities produced by the quadruplexes formed with the 1 bp mismatched dsDNA targets plus the dsDNA probe (SEQ ID NO:3), ranged from 17.0% to 34.6% less than that achieved by the perfectly matched quadruplexes. When the maximum fluorescent intensity values were normalized by subtracting the fluorescent intensity value obtained for the dsDNA probe (SEQ ID NO:3), the mismatched quadruplexes comprised of SEQ ID NO:5+SEQ ID NO:3, SEQ ID NO:6+SEQ ID NO:3, SEQ ID NO:7+SEQ ID NO:3 or SEQ ID NO:8+SEQ ID NO:3 emitted fluorescent intensities that were 45.0%, 49.4%, 32.9% and 66.8% lower, respectively, than that achieved by the perfectly homologous quadruplexes (SEQ ID NO:4+SEQ ID NO:3) in the untreated medium (FIG. 8C). The dsDNA target plus 100 nM YOYO-1 control samples showed similar levels of fluorescence, which constituted no more than 17.4% of the fluorescent level achieved by the perfectly matched quadruplex (FIG. 8C).

Electrical pretreatment with forty 9V pulses to the test medium slightly reduced perfectly homologous quadruplex (SEQ ID NO:4+SEQ ID NO:3) formation (FIG. 8D), resulting in a 4.2% decrease in fluorescent emission compared to that observed with the same sample in untreated test medium (FIG. 8C). The application of forty 9V pulses to the test medium prior to incubation had a distinct effect on mismatched parallel homologous quadruplex formation, resulting in significant increases in binding signal as compared to that observed from the same samples reacted in the untreated medium. The fluorescent intensities emitted by the 1 bp mismatched complexes in the pretreated medium were 1.4% to 38.9% lower than that obtained by the perfectly matched complexes (FIG. 8D). When the maximum fluorescent intensity values were normalized by subtracting the fluorescent intensity value obtained for the dsDNA probe (SEQ ID NO:3), the mismatched quadruplexes comprised of SEQ ID NO:5+SEQ ID NO:3, SEQ ID NO:6+SEQ ID NO:3, SEQ ID NO:7+SEQ ID NO:3 or SEQ ID NO:8+SEQ ID NO:3 emitted fluorescent intensities that were 18.1%, 2.8%, 22.8% and 77.3% lower, respectively, than that achieved by the perfectly homologous quadruplexes (SEQ ID NO:4+SEQ ID NO:3) in the pretreated medium (FIG. 8D). Control samples comprising each dsDNA target plus 100 nM YOYO-1 in pretreated medium exhibited similar levels of fluorescence that constituted no more than 20.2% of the fluorescent level achieved by the perfectly homologous quadruplex (FIG. 8D).

Electrical pretreatment of the test medium noticeably diminished the specificity of parallel homologous quadruplex binding, while significantly increasing the specificity of nested complementary quadruplex binding.

### Example 9

Example 9 applies the method of electrical pretreatment of medium to Watson-Crick triplex DNA binding between non-amplified, non-denatured genomic Drosophila dsDNA targets and ssDNA probes of mixed base sequence. The Drosophila genome at 165×10⁶ bp approximates 5% of the human genome, a genomic size slightly greater than that of an average human chromosome.

Wild-type genomic Drosophila melanogaster DNA (wt gDNA) and mutant genomic Drosophila melanogaster DNA (ts1 gDNA), that contained a 1 bp A to T transition within the erg gene (a seizure locus) [J. Neuroscience 17, 875-881 (1997)] was purified by numerous phenol-chloroform extractions from Drosophila flies and kindly supplied by Dr. Barry Ganetzky (University of Wisconsin, Madison, Wis., U.S.A.). Wild-type 15-mer ssDNA probes designed to be perfectly complementary to the sense or antisense strand of the wt gDNA around the mutation site were synthesized and purified by HPLC as above. SsDNA oligonucleotides were diluted in ddH₂O to a concentration of 1 pmole/μl.

Sequence for the antisense strand of the wild-type Drosophila 15-mer DNA probe (SEQ ID NO:10): 5′-CTG CCC CTT CGG CTC-3′

Forty 500 msec pulses of nine volts of DC current, separated by 10 second intervals were applied to 50 μl of test medium consisting of 1.0×TBE. Immediately after the final pulse of DC current, five pmoles of the 15-mer ssDNA probe (antisense strand of SEQ ID NO:10), 50 ng of the genomic Drosophila dsDNA target and YOYO-1 were added to the untreated and electrically pretreated test media to give a final volume of 100 μl. The final buffer concentration was 0.5×TBE, and the final YOYO-1 concentration was 500 nM. Samples were incubated at room temperature for 5 min after addition of the DNA reagents, placed into a 3 mm quartz cuvette and irradiated for 40 msec with an argon ion laser beam having a wavelength of 488 nm. The fluorescence emission was monitored at various timepoints up to 60 min. Serial emission measurements were acquired. The intensity of fluorescence was plotted as a function of incubation time for each sample analyzed.

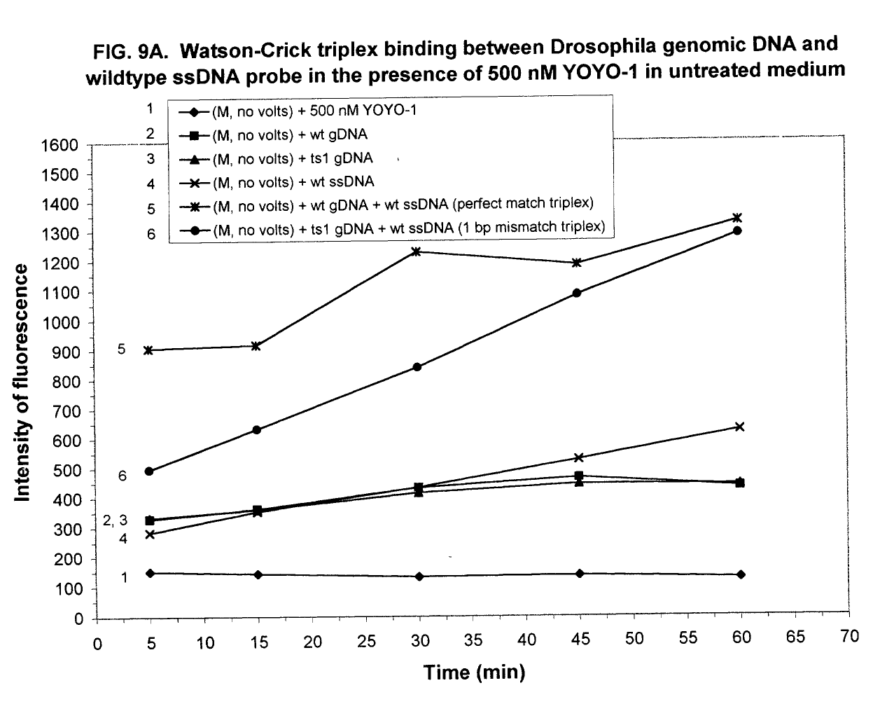

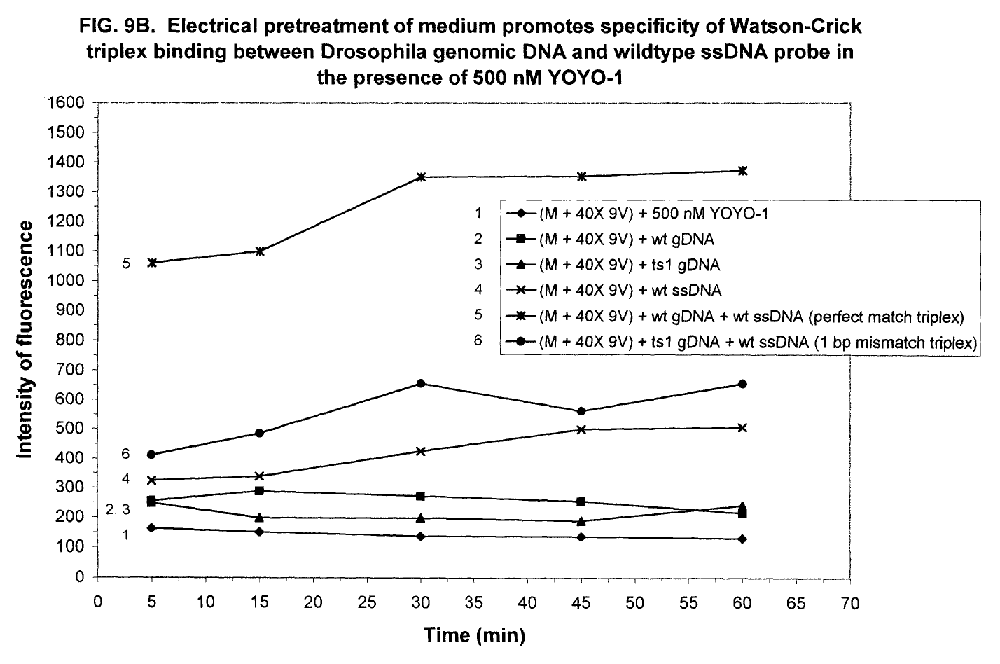

When the wild-type 15-mer ssDNA probe (antisense strand of SEQ ID NO:10) was reacted with the wild-type gDNA target and the mutant tsl gDNA target, dsDNA:ssDNA complexes were formed in the untreated medium at room temperature under non-denaturing conditions (FIG. 9A). Perfectly matched DNA complexes (antisense strand of SEQ ID NO:10+wt gDNA) emitted the highest fluorescent intensity at each time point assayed from 5 min to 60 minutes after addition of the DNA. Incompletely complementary complexes with a 1 bp T-T mismatch (antisense strand of SEQ ID NO:10+ts1 gDNA) produced a fluorescent intensity that was 54.4% lower than that observed with the perfectly matched complexes after a 5 min reaction in the untreated medium (FIG. 9A). The level of discrimination between the fluorescence emitted by the perfectly matched complexes and that emitted by the 1 bp T-T mismatched complexes decreased with time, such that after a 60 minute incubation in the untreated medium, the 1 bp mismatched complexes produced a fluorescent intensity that was only 3.6% lower than that observed with the perfectly matched complexes (FIG. 9A).

Control samples comprising each gDNA target plus 500 nM YOYO-1 or the ssDNA probe (antisense strand of SEQ ID NO:10) plus 500 nM YOYO-1 exhibited similar levels of fluorescence, which constituted no more than 24.0% of the fluorescent level achieved by the perfectly matched complexes (FIG. 9A). When the maximum fluorescent intensity values were normalized to account for triplex formation only, by subtracting the values of fluorescent intensities obtained for each gDNA target alone, the 1 bp T-T mismatched dsDNA:ssDNA triplexes produced fluorescent intensities that were 71.8% and 5.6% lower than that achieved by the perfectly matched triplexes after a 5 min and a 60 min incubation, respectively.

Electrical pretreatment with forty 9V pulses to the test medium enhanced perfectly matched Watson-Crick triplex (antisense strand of SEQ ID NO:10+wt gDNA) formation, resulting in a 19.1% increase in fluorescent emission at the 5 min timepoint when compared to that observed with the same sample in untreated test medium (FIG. 9B). Mutant ts1 gDNA formed measurably fewer triplex complexes with the ssDNA probe (antisense strand of SEQ ID NO:10), than did the fully complementary wild-type gDNA target at each timepoint assayed in the electrically pretreated medium (FIG. 9B). The fluorescent intensity emitted by the 1 bp T-T mismatched complexes (antisense strand of SEQ ID NO:10+ts1 gDNA) in the pretreated medium was 72.3% lower than that obtained by the perfectly matched complexes (antisense strand of SEQ ID NO:10+wt gDNA) after a 5 min incubation. This high level of specificity of binding was maintained over time. Even 60 min after electrical pretreatment of the medium and addition of the DNA, the 1 bp T-T mismatched complexes produced a fluorescent intensity that was 57.7% lower than that emitted by the perfectly matched complexes at that timepoint (FIG. 9B).

Control samples comprising each gDNA target plus 500 nM YOYO-1 exhibited levels of fluorescence, which were above that obtained by YOYO-1 alone in the pretreated medium (FIG. 9B). The level of fluorescence generated by the ssDNA probe plus 500 nM YOYO-1 was slightly greater, but still only 18.0% of the level produced by the perfectly matched complexes at the 5 min timepoint. When the maximum fluorescent intensity values were normalized to account for triplex formation only, by subtracting the values of fluorescent intensities obtained for each gDNA target, the 1 bp T-T mismatched gDNA:ssDNA tripiexes produced fluorescent intensities that were 79.8% and 64.2% lower than that achieved by the perfectly matched triplexes after a 5 min and a 60 min incubation in the pretreated medium, respectively (FIG. 9B).

In the absence of electrical pretreatment of the medium, clear discrimination between perfectly complementary gDNA:ssDNA triplexes and 1 bp mismatched gDNA:ssDNA triplexes was achieved after just 5 minutes of incubation in the presence of YOYO-1. Electrical pretreatment of the test medium significantly enhanced the specificity of Watson-Crick triplex binding between non-amplified, non-denatured genomic Drosophila dsDNA and ssDNA of mixed base sequence. These results demonstrate that the triplex assay employed is sufficiently sensitive to readily detect 1 bp mutations in non-denatured genomic dsDNA samples, eliminating the necessity for PCR amplification of target.

While the invention has been described in detail and with reference to specific examples thereof, it will be apparent to one skilled in the art that various changes and modifications can be made without departing from the spirit and scope thereof.

What is claimed is:

1. A method for influencing binding of a first biopolymer to a second biopolymer, said method comprising applying an electric charge to a binding medium in which the first and second biopolymers are to be bonded together, wherein the electric charge is applied sufficiently to enhance or diminish a binding characteristic of the binding to thereby influence the binding, provided that the binding characteristic is not denaturation of the first and second biopolymers from each other or from another biopolymer.

2. The method of claim 1, wherein the first and second biopolymers are members independently selected from the group consisting of nucleic acids and analogues thereof.

3. The method of claim 2, wherein the binding characteristic is a binding affinity between the first and second biopolymers, and the binding affinity is increased by the electric charge.

4. The method of claim 2, wherein the binding characteristic is a binding affinity between at least one of the first and second biopolymers and an object other than the first and second biopolymers, and the binding affinity is reduced by the electric charge.

5. The method of claim 2, wherein the binding characteristic is a binding rate, and the binding rate is increased by the electric charge.

6. The method of claim 1, wherein the first and second biopolymers are nucleic acids or nucleic acid analogues capable of binding together according to a Watson-Crick binding motif and capable of binding together according to a homologous binding motif, and the binding characteristic is a binding motif preference.

7. The method of claim 6, wherein the electric charge shifts the binding motif preference towards the homologous binding motif sufficiently that the first and second biopolymers are solely or predominantly bound according to the homologous binding motif.

8. The method of claim 6, wherein the electric charge shifts the binding motif preference towards the Watson-Crick binding motif sufficiently that the first and second biopolymers are solely or predominantly bound according to the Watson-Crick binding motif.

9. The method of claim 2, wherein the electric charge is only applied to the binding medium prior to introducing the first and second biopolymers to the binding medium.

10. The method of claim 9, wherein the introducing of the first and second biopolymers to the binding medium follows the electric charge applying by 5 to 40 minutes.

11. The method of claim 2, wherein the electric charge is only applied to the binding medium when only one of the first and second biopolymers is present in the binding medium.

12. The method of claim 2, wherein the electric charge is only applied to the binding medium when both the first and second biopolymers are present in the binding medium.

13. The method of claim 2, wherein the electric charge is only applied to the binding medium in a first vessel, the binding medium is transferred from the first vessel to a second vessel, and the first and second biopolymers are bonded together in the second vessel.

14. The method of claim 2, wherein the electric charge is applied to only a portion of the binding medium.

15. The method of claim 2, wherein the electric charge is applied to all of the binding medium.

16. The method of claim 2, wherein the electric charge is DC electrical current.

17. The method of claim 2, wherein the electric charge is AC electrical current.

18. The method of claim 2, wherein the electric charge is applied in a single application.

19. The method of claim 2, wherein the electric charge is applied in a plurality of pulses.

20. The method of claim 19, wherein the pulses are of identical or varied currents, are applied for identical or varied durations and/or are applied at identical or varied intervals.

21. The method of claim 19, wherein the pulses are DC pulses from 1 V to 27 V.

22. The method of claim 19, wherein from 2 to 100 of the pulses are applied to the binding medium.

23. The method of claim 2, wherein the electric charge is applied to the binding medium for less than 1 hour.

24. The method of claim 2, wherein the electric charge is applied to the binding medium while the first and second biopolymers are binding together.

25. The method of claim 2, wherein the electric charge is applied to the binding medium after the first and second biopolymers have bonded together.

26. The method of claim 2, wherein the first and second biopolymers are free of any solid support.

27. The method of claim 2, wherein at least one of the first and second biopolymers is bound to a solid support.

28. The method of claim 2, wherein one of the first and second biopolymers is a nucleic acid and the other of the first and second biopolymers is a nucleic acid analogue.

29. The method of claim 28, wherein the nucleic acid is genomic DNA or RNA.

30. The method of claim 28, wherein the nucleic acid analogue is PNA or LNA.

31. The method of claim 2, wherein the first and second biopolymers bind without denaturing to form a triplex or a quadruplex.

32. The method of claim 1, wherein the binding medium moves through a semi-permeable apparatus, capillary or channel.

33. The method of claim 1, wherein the binding characteristic is neither enhanced nor diminished as a result of electrophoretic migration of the biopolymers or electroporation through a membrane separating the biopolymers.

34. The method of claim 1, wherein the binding medium achieves greater organization than that of a bulk solution or a bulk medium.

35. The method of claim 2, wherein the binding characteristic comprises a binding affinity between the first and second biopolymers, and the method further comprises determining an identity of the second biopolymer as a function of the binding affinity.

36. The method of claim 35, wherein the identity determining comprises determining the binding affinity by analyzing a signal emitted from the binding medium.

37. The method of claim 36, wherein the signal is an intensity of a fluorescent emission from fluorescent labels in the binding medium.

38. The method of claim 36, wherein a signal to noise ratio of the signal is increased by the application of the electric charge.

39. The method of claim 35, wherein the identity is determined one second to 45 minutes after applying the electric charge to the binding medium.

40. The method of claim 35, wherein the identity determining comprises comparing fluorescent emission intensities from fluorescent labels in the binding medium before and after applying the electric charge.

41. The method of claim 2, wherein the influenced binding has a therapeutic effect on an organism.

42. The method of claim 2, wherein the influenced binding facilitates construction of an engineered structure.

43. The method of claim 1, wherein one of the first and second biopolymers is a nucleic acid or nucleic acid analogue and the other of the first and second biopolymers comprises amino acids or amino acid analogues.

44. The method of claim 1, wherein each of the first and second biopolymers comprises amino acids or amino acid analogues.

45. An apparatus for practicing the method of claim 1, said apparatus comprising a binding medium container, electrodes adapted to contact the binding medium in the binding medium container, a voltage source to apply the electric charge to the binding medium in the binding medium container and a detector.


SEQUENCE LISTING (10 sequences)

SEQ ID NO: 1 - DNA, 15 bp, Artificial Sequence
  Derived from Exon 10 of Human Cystic Fibrosis Gene
ctgtcatctctggtg

SEQ ID NO: 2 - DNA, 15 bp, Artificial Sequence
  Derived from Exon 10 of Human Cystic Fibrosis Gene
caccagagatgacag

SEQ ID NO: 3 - DNA, 15 bp, Artificial Sequence
  Derived from Exon 10 of Human Cystic Fibrosis Gene
ctgtcatctctggtg

SEQ ID NO: 4 - DNA, 50 bp, Artificial Sequence
  Derived from Exon 10 of Human Cystic Fibrosis Gene
gagcaccatgacagacactgtcatctctggtgtgtcctacgatgactctg

SEQ ID NO: 5 - DNA, 50 bp, Artificial Sequence
  Derived from Exon 10 of Human Cystic Fibrosis Gene
gagcaccatgacagacactgtcatctttggtgtgtcctacgatgactctg

SEQ ID NO: 6 - DNA, 50 bp, Artificial Sequence
  Derived from Exon 10 of Human Cystic Fibrosis Gene
gagcaccatgacagacactgtcttctctggtgtgtcctacgatgactctg

SEQ ID NO: 7 - DNA, 50 bp, Artificial Sequence
  Derived from Exon 10 of Human Cystic Fibrosis Gene
gagcaccatgacagacactgtcatccctggtgtgtcctacgatgactctg

SEQ ID NO: 8 - DNA, 50 bp, Artificial Sequence
  Derived from Exon 10 of Human Cystic Fibrosis Gene
gagcaccatgacagacactgtcgtctctggtgtgtcctacgatgactctg

SEQ ID NO: 9 - DNA, 15 bp, Artificial Sequence
  Derived from Exon 10 of Human Cystic Fibrosis Gene
gacagtagagaccac

SEQ ID NO: 10 - DNA, 15 bp, Artificial Sequence
  Derived from Drosophila melanogaster DNA
ctgccccttcggctc